(19) United States
(12) Patent Application Publication
(10) Pub. No.: US 2007/0128592 A1
(43) Pub. Date: Jun. 7, 2007

(54) Method and nucleic acids for the analysis of a lung cell proliferative disorder

(75) Inventors: Matthias Burger, Berlin (DE); John K. Field, Wirral (IE); Bulent Genc, Berlin (DE); Triantafillos Liloglou, Liverpool (GB); Evelyne Lipscher, Berlin (DE); Sabine Maier, Berlin (DE); Inko Nimmrich, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 10/498,430

(22) PCT Filed: Dec. 10, 2002
(86) PCT No.: PCT/EP02/14026

§ 371 Date: Dec. 21, 2005

(30) Foreign Application Priority Data
Dec. 14, 2001 (DE) 101 61 625.2

(51) Int. Cl. C12Q 1/68

(52) U.S. Cl. 435/6

(57) ABSTRACT

The present invention relates to modified and genomic sequences, to oligonucleotides and/or PNA-oligomers for detecting the cytosine methylation state of genomic DNA, as well as to a method for ascertaining genetic and/or epigenetic parameters of genes for use in the differentiation, diagnosis, treatment and/or monitoring of lung cell proliferative disorders, or the predisposition to lung cell proliferative disorders.

### FIELD OF THE INVENTION

The levels of observation that have been studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The present invention relates to nucleic acids, oligonucleotides, PNA-oligomers, and to a method for the analysis of lung cell proliferative disorders, the differentiation between subclasses of said disorder or the detection of a predisposition to said disorders, by analysis of the genetic and/or epigenetic parameters of genomic DNA and, in particular, with the cytosine methylation status thereof.

Lung cancer is among the most commonly occurring malignancies in the world and is one of the few that continues to show an increasing incidence. In men it is the leading cause of of death in Western countries. In 2000, the incidence in the US is estimated to be 164 000 new cases and 157 000 deaths from the disease. 5 year survival rates are only 14% in the US (Ginsberg et al., Principles & Practice of Oncology. 6^{th} Edition). The most prominent risk factor is smoking, around 80% of lung cancer deaths among men and 75% among women are likely to be attributable to smoking (Minna et al., Cancer: principles and practice of oncology, 3^{rd} ed., 1989).

Lung cancer falls into two major histologic classes, small cell lung cancer and non-small cell lung cancer. The latter one represents 82% of lung cancer cases (Murren et al., Principles & Practice of Oncology. 6^{th} Edition) and can be further subclassified into squamous cell carcinoma, once the most frequent of all lung cancers in North America, and adenocarcinoma, to which 40% of new lung cancer cases can be attributed (Ginsberg et al., Principles & Practice of Oncology. 6^{th} Edition). Squamous cell carcinoma arises most frequently in the proximal segmental bronchi. Because of the ability of squamous cells to exfoliate, this tumour can be detected by cytologic examination of sputum. Adenocarcinoma usually arises more peripherally and has a somewhat worse prognosis compared to squamous cell carcinoma.

Because of the poor prognosis of lung cancer, identification of patients at an early stage, where the disease can still be cured, is of outstanding importance. Currently, most patients present with metastatic (stage IV) disease (Ginsberg et al., Principles & Practice of Oncology. 6^{th} Edition). Sputum or bronchoalveolar lavage analysis, imaging techniques from conventional chest radiography to spiral computed tomography, percutaneous fine-needle aspiration, bronchoscopy are used to diagnose patients in whom the disease is suspected. Whereas helical computed tomographic scans are particularly successful in picking up small peripheral adenocarcinomas that cannot yet be visualised by standard chest x-rays, cytologic examination of sputum provides a high sensitivity for central squamous cell lesions. However, because of their invasiveness, radiation exposure and, above all, the high number of false positives, these methods are currently only applied in a very small subset of individuals known to be at high risk for the disease or if symptoms are already present.

In the last decade, knowledge has accumulated on molecular alterations which occur during progression from dysplasia or atypia to cancerous lesions of the lung. These alterations include chromosomal abnormalities such as deletions of 3p, 9p and 17p (Sekido et al., Principles & Practice of Oncology. 6^{th} Edition), microsatellite instability (Sekido et al., Biochim Biophys Acta 1998, 1378: F21), activation of protooncogenes, e.g. EGFR, ERBB2, KIT, and MET (Rusch et al., Clin Cancer Res 1997, 3:515, Tsai et al., Cancer Res 1996, 56:206, Krystal et al., Cancer Res 1998, 58:4660), inactivation of tumor suppressor genes like p53 (Bennett et al., J Pathol 1999, 187:8), p16 (Sekido et al., Biochim Biophys Acta 1998, 1378: F21, Belinsky et al., PNAS USA 1998, 95: 11891) and RB (Reissmann et al., Oncogene 1993, 8:1913). One of the earliest molecular alterations in tumorigenesis is aberrant DNA methylation. In a recent study, Dai and coworkers were able to show that out of 1184 CpG islands screened by RLGS analysis up to 5.3% are methylated in some non-small cell lung cancers. In addition, aberrant methylation could be detected not only in the tumour itself, but also in different body fluids, such as serum (Esteller et al., Cancer Res, 1999, 59:67) and bronchoalveolar lavage samples (Ahrendt et al., J Natl Cancer Inst 91:332).

Molecular markers offer the advantage that even samples of very small sizes and samples whose tissue architecture has not been maintained, e.g. very small biopsies or single cells can be analysed quite efficiently. In addition, molecular alterations identified in different tumour types can be detected also in body fluids such as serum, plasma, sputum or bronchoalveolar lavage, probably much earlier than cytological analysis. Detailed knowledge of the molecular pathogenesis of a disease also offers the possibility to develop new drugs targeted specifically at alterations occurring at a specific stage in the disease.

Aberrant DNA methylation within CpG islands is common in human malignancies leading to abrogation or overexpression of a broad spectrum of genes (Jones, P. A. Cancer Res 65:2463-2467, 1996). Abnormal methylation has also been shown to occur in CpG rich regulatory elements in intronic and coding parts of genes for certain tumours (Chan, M. F., et al., Curr Top Microbiol Immunol 249:75-86,2000). Highly characteristic DNA methylation patterns could also be shown for breast cancer cell lines (Huang, T. H.-M., et al., Hum Mol Genet 8:459-470, 1999). Large-scale methylation analysis has not been applied to lymphomas so far, but alterations of the methylation of single genes have been described in several subtypes of Non-Hodgkin lymphoma, e.g. TCL1 (Yuille et al., Genes Chromosomes Cancer 2001, 30:336-41), p15 and AR (Baur et al., Blood. 1999, 94:1773-81, Martinez-Delgado et al., Leukemia 1998 12:937-41), the androgen receptor (McDonald et al., Genes Chromosomes Cancer. 2000 28:246-57), and the MyoD1 gene (Taylor et al., Leukemia. 2001, 15:583-9).

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

A relatively new and currently the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions.

Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 Dec. 15; 24(24):5064-6). Using this method, it is possible to analyse individual cells, which illustrates the potential of the method. However, currently only individual regions of a length of up to approximately 3000 base pairs are analyzed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April; 5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November; 17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15; 25(12):2529-31, WO 95/00669) or by enzymatic digestion (Xiong Z, Laird P W. COBRA: a sensitive and quantitative DNA methylation assay. Nucleic Acids Res. 1997 Jun. 15; 25(12):2532-4). In addition, detection by hybridisation has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet 1997 March; 6(3):387-95; Feil R, Charlton J, Bird A P, Walter J, Reik W. Methylation analysis on individual chromosomes: improved protocol for bisulphite genomic sequencing. Nucleic Acids Res. 1994 Feb. 25; 22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19; 157(1-2):261-4; WO 97/46705, WO 95/15373, and WO 97/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionisation of proteins with molecular masses exceeding 10,000 daltons. Anal Chem. 1988 Oct. 15; 60(20):2299-301). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystalisation. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25; 23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

### DESCRIPTION

The invention provide a method for the analysis of biological samples for features associated with the development of lung cell proliferative disorders, characterised in that the nucleic acid of at least one member of the group comprising MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1 is/are contacted with a reagent or series of reagents capable of distinguishing between methylated and non methylated CpG dinucleotides within the genomic sequence of interest.

The present invention makes available a method for ascertaining genetic and/or epigenetic parameters of genomic DNA. The method is for use in the improved diagnosis, treatment and monitoring of lung cell proliferative disorders, more specifically by enabling the improved identification of and differentiation between subclasses of said disorder and the genetic pre-disposition to said disorders. The invention presents improvements over the state of the art in that it enables a highly specific classification of lung carcinomas, thereby allowing for improved and informed treatment of patients.

In a particularly preferred embodiment the present invention makes available methods and nucleic acids that allow the differentiation between squamous cell carcinoma, and adenocarcinoma and their respective adjacent lung tissues.

Furthermore, the method enables the analysis of cytosine methylations and single nucleotide polymorphisms.

In a preferred embodiment, the method comprises the following steps:

In the first step of the method the genomic DNA sample must be isolated from tissue or cellular sources. Such sources may include lung tissue samples, cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the next step of the method, this may be by any means standard in the state of the art, in particular, but not limited to, with restriction endonucleases.

In the second step of the method, the genomic DNA sample is treated in such a manner that cytosine bases which are unmethylated at the 5′-position are converted to uracil, thymidine, or another base which is dissimilar to cytosine in terms of hybridisation behaviour. This will be understood as ‘pretreatment’ hereinafter.

The above described treatment of genomic DNA is preferably carried out with bisulfite (sulfite, disulfite) and subsequent alkaline hydrolysis which results in a conversion of non-methylated cytosine nucleobases to uracil or to another base which is dissimilar to cytosine in terms of base pairing behaviour. If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines.

Fragments of the pretreated DNA are amplified, using sets of primer oligonucleotides according to SEQ ID NO: 308 to SEQ ID NO: 427, and a, preferably heat-stable, polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

The method may also be enabled by the use of alternative primers, the design of such primers is obvious to one skilled in the art. These should include at least two oligonucleotides whose sequences are each reverse complementary or identical to an at least 18 base-pair long segment of the base sequences specified in the appendix (SEQ ID NO:76 to SEQ ID NO: 307). Said primer oligonucleotides are preferably characterised in that they do not contain any CpG dinucleotides. In a particularly preferred embodiment of the method, the sequence of said primer oligonucleotides are designed so as to selectively anneal to and amplify, only the lung tissue specific DNA of interest, thereby minimising the amplification of background or non relevant DNA. In the context of the present invention, background DNA is taken to mean genomic DNA which does not have a relevant tissue specific methylation pattern, in this case, the relevant tissue being lung, both healthy and diseased.

According to the present invention, it is preferred that at least one primer oligonucleotide is bound to a solid phase during amplification. The different oligonucleotide and/or PNA-oligomer sequences can be arranged on a plane solid phase in the form of a rectangular or hexagonal lattice, the solid phase surface preferably being composed of silicon, glass, poly-styrene, aluminium, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualised by means of matrix assisted laser desorption/ionisation mass spectrometry (MALDI) or using electron spray mass spectrometry (ESI).

The amplificates obtained in the second step of the method are subsequently hybridised to an array or a set of oligonucleotides and/or PNA probes. In this context, the hybridisation takes place in the manner described as follows. The set of probes used during the hybridisation is preferably composed of at least 10 oligonucleotides or PNA-oligomers. In the process, the amplificates serve as probes which hybridise to oligonucleotides previously bonded to a solid phase. In a particularly preferred embodiment, the oligonucleotides are taken from the group comprising SEQ ID NO: 428 to SEQ ID NO: 917. In a further preferred embodiment the oligonucleotides are taken from the group comprising SEQ ID NO: 884 to SEQ ID NO: 917. The non-hybridised fragments are subsequently removed. Said oligonucleotides contain at least one base sequence having a length of 10 nucleotides which is reverse complementary or identical to a segment of the base sequences specified in the appendix, the segment containing at least one CpG or TpG dinucleotide. In a further preferred embodiment the cytosine of the CpG dinucleotide, or in the case of TpG, the thymidine, is the 5^{th} to 9^{th} nucleotide from the 5′-end of the 10-mer. One oligonucleotide exists for each CpG or TpG dinucleotide.

In the fifth step of the method, the non-hybridised amplificates are removed.

In the final step of the method, the hybridised amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualised by means of matrix assisted laser desorption/ionisation mass spectrometry (MALDI) or using electron spray mass spectrometry (ESI). The produced fragments may have a single positive or negative net charge for better detectability in the mass spectrometer.

The aforementioned method is preferably used for ascertaining genetic and/or epigenetic parameters of genomic DNA.

In order to enable this method, the invention further provides the modified DNA of genes MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1 as well as oligonucleotides and/or PNA-oligomers for detecting cytosine methylations within said genes. The present invention is based on the discovery that genetic and epigenetic parameters and, in particular, the cytosine methylation patterns of genomic DNA are particularly suitable for improved diagnosis, treatment and monitoring of lung cell proliferative disorders. Furthermore, the invention enables the differentiation between different subclasses of lung carcinomas or detection of a predisposition to lung carcinomas.

The nucleic acids according to the present invention can be used for the analysis of genetic and/or epigenetic parameters of genomic DNA.

This objective is achieved according to the present invention using a nucleic acid containing a sequence of at least 18 bases in length of the pretreated genomic DNA according to one of SEQ ID NO: 76 through SEQ ID NO: 307 and sequences complementary thereto.

The modified nucleic acid could heretofore not be connected with the ascertainment of disease relevant genetic and epigenetic parameters.

The object of the present invention is further achieved by an oligonucleotide or oligomer for the analysis of pretreated DNA, for detecting the genomic cytosine methylation state, said oligonucleotide containing at least one base sequence having a length of at least 10 nucleotides which hybridises to a pretreated genomic DNA according to SEQ ID NO: 76 to SEQ ID NO: 307. The oligomer probes according to the present invention constitute important and effective tools which, for the first time, make it possible to ascertain specific genetic and epigenetic parameters during the analysis of biological samples for features associated with the development of lung cell proliferative disorders. Said oligonucleotides allow the improved diagnosis, treatment and monitoring of lung cell proliferative disorders and detection of the predisposition to said disorders. Furthermore, they allow the differentiation of different subclasses of lung carcinomas. The base sequence of the oligomers preferably contains at least one CpG or TpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5^{th}-9^{th} nucleotide from the 5′-end of the 13-mer; in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least one oligomer for each of the CpG dinucleotides within SEQ ID NO: 76 to SEQ ID NO: 307. Preferred is a set which contains at least one oligomer for each of the CpG dinucleotides, from SEQ ID NO: 428 to SEQ ID NO: 917. Further preferred is a set comprising SEQ ID NO: 884 to SEQ ID NO: 917.

In the case of the sets of oligonucleotides according to the present invention, it is preferred that at least one oligonucleotide is bound to a solid phase. It is further preferred that all the oligonucleotides of one set are bound to a solid phase.

The present invention moreover relates to a set of at least 10 n (oligonucleotides and/or PNA-oligomers) used for detecting the cytosine methylation state of genomic DNA using treated versions of said genomic DNA (according to SEQ ID NO: 76 to SEQ ID NO: 307 and sequences complementary thereto). These probes enable improved diagnosis, treatment and monitoring of lung cell proliferative disorders. In particular they enable the differentiation between different sub classes of lung cell proliferative disorders and the detection of a predisposition to said disorders. In a particularly preferred embodiment the set comprises SEQ ID NO: 59 to SEQ ID NO: 917.

The set of oligomers may also be used for detecting single nucleotide polymorphisms (SNPs) using pretreated genomic DNA according to one of SEQ ID NO: 76 to SEQ ID NO: 307.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are suitable alternatives.

Therefore, a further subject matter of the present invention is a method for manufacturing an array fixed to a carrier material for the improved diagnosis, treatment and monitoring of lung cell proliferative disorders, the differentiation between different subclasses of lung carcinomas and/or detection of the predisposition to lung cell proliferative disorders. In said method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the improved diagnosis, treatment and monitoring of lung cell proliferative disorders. Furthermore the DNA chip enables detection of the predisposition to lung cell proliferative disorders and the differentiation between different subclasses of lung carcinomas. The DNA chip contains at least one nucleic acid according to the present invention. DNA chips are known, for example, in U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond or are complementary to a 18 base long segment of the base sequences specified in the appendix (SEQ ID NO: 76 to SEQ ID NO: 307), oligonucleotides and/or PNA-oligomers as well as instructions for carrying out and evaluating the described method. However, a kit along the lines of the present invention can also contain only part of the aforementioned components.

The oligomers according to the present invention or arrays thereof as well as a kit according to the present invention are intended to be used for the improved diagnosis, treatment and monitoring of lung cell proliferative disorders. Furthermore the use of said inventions extends to the differentiation between different subclasses of lung carcinomas and detection of the predisposition to lung cell proliferative disorders. According to the present invention, the method is preferably used for the analysis of important genetic and/or epigenetic parameters within genomic DNA, in particular for use in improved diagnosis, treatment and monitoring of lung cell proliferative disorders, detection of the predisposition to said disorders and the differentiation between subclasses of said disorders.

The methods according to the present invention are used, for example, for improved diagnosis, treatment and monitoring of lung cell proliferative disorders progression, detection of the predisposition to said disorders and the differentiation between subclasses of said disorders.

A further embodiment of the invention is a method for the analysis of the methylation status of genomic DNA without the need for pretreatment. In the first step of the method the genomic DNA sample must be isolated from tissue or cellular sources. Such sources may include cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the treatment, this may be any means standard in the state of the art, in particular with restriction endonucleases. In the second step, the DNA is then digested with one or more methylation sensitive restriction enzymes. The digestion is carried out such that hydrolysis of the DNA at the restriction site is informative of the methylation status of a specific CpG dinucleotide.

In the third step the restriction fragments are amplified. In a preferred embodiment this is carried out using a polymerase chain reaction.

In the final step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

The present invention moreover relates to the diagnosis and/or prognosis of events which are disadvantageous or relevant to patients or individuals in which important genetic and/or epigenetic parameters within genomic DNA, said parameters obtained by means of the present invention may be compared to another set of genetic and/or epigenetic parameters, the differences serving as the basis for the diagnosis and/or prognosis of events which are disadvantageous or relevant to patients or individuals.

In the context of the present invention the term “hybridisation” is to be understood as a bond of an oligonucleotide to a completely complementary sequence along the lines of the Watson-Crick base pairings in the sample DNA, forming a duplex structure.

In the context of the present invention, “genetic parameters” are mutations and polymorphisms of genomic DNA and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further modifications of DNA bases of genomic DNA and sequences further required for their regulation. Further epigenetic parameters include, for example, the acetylation of histones which, cannot be directly analysed using the described method but which, in turn, correlates with the DNA methylation.

In the following, the present invention will be explained in greater detail on the basis of the figures, sequences and examples without being limited thereto.

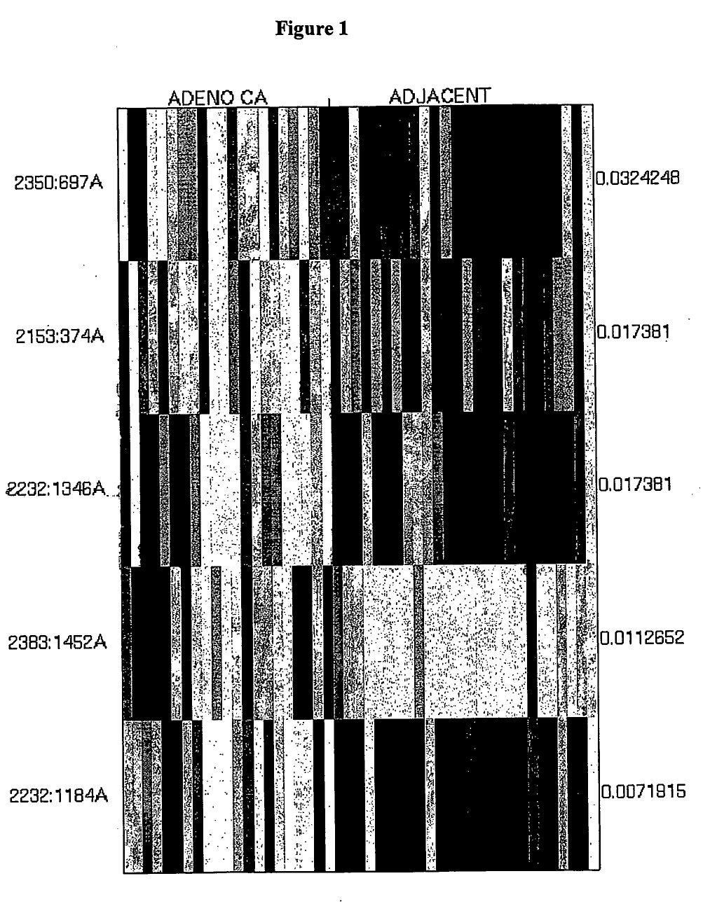

FIG. 1

FIG. 1 shows the differentiation between adenocarcinoma and adjacent tissues according to Example 2. The labels on the left side of the plot are gene and CpG identifiers, these can be cross referenced in Table 3. The labels on the right side give the significance (p-value, T-test) of the difference between the means of the two groups. Each row corresponds to a single CpG and each column to the methylation levels of one sample. CpGs are ordered according to their contribution to the differentiation between the two tissue types with increasing contribution from top to bottom. Black indicates total methylation at a given CpG position, white represents no methylation at the particular position, with degrees of methylation represented in grey, from light (low proportion of methylation) to dark (high proportion of methylation).

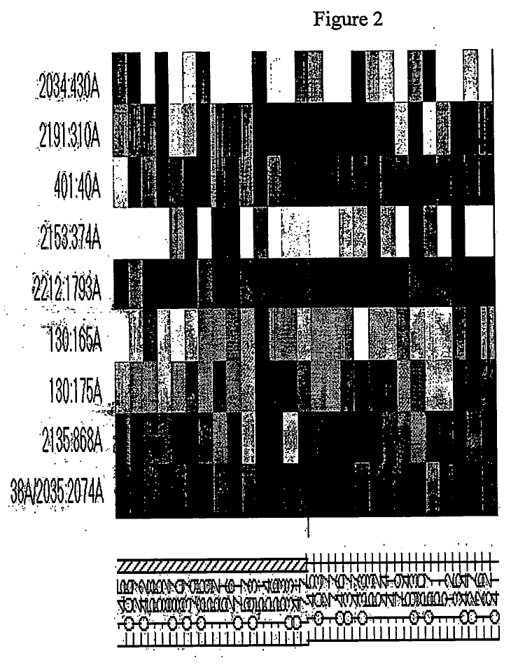

FIG. 2

FIG. 2 shows the differentiation of squamous cell carcinoma tissue from adjacent tissues using informative CpG-Positions from 9 genes. Informative CpG-Positions are further described in Table 4. P-values are obtained using the Wilcoxon test. The labels on the left side of the plot are gene and CpG identifiers, these can be cross referenced in Table 4. The labels on the right side give the significance (p-value, T-test) of the difference between the means of the two groups. Each row corresponds to a single CpG and each column to the methylation levels of one sample. CpGs are ordered according to their contribution to the differentiation between the two tissue types with increasing contribution from top to bottom. Black indicates total methylation at a given CpG position, white represents no methylation at the particular position, with degrees of methylation represented in grey, from light (low proportion of methylation) to dark (high proportion of methylation).

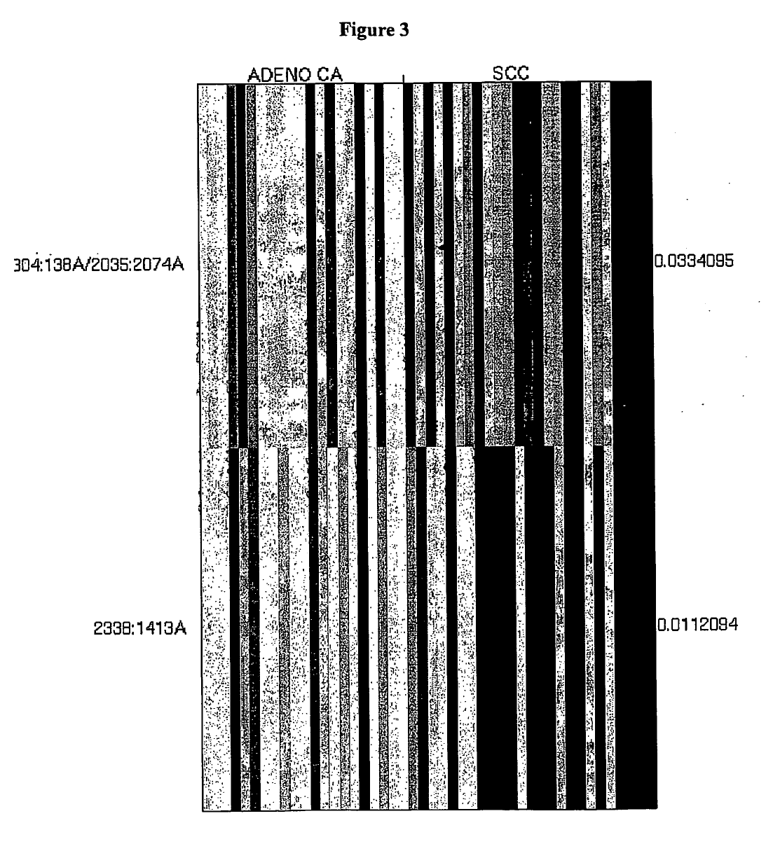

FIG. 3

FIG. 3 shows the differentiation between adenocarcinoma and squamous cell carcinoma according to Example 2. The labels on the left side of the plot are gene and CpG identifiers, these can be cross referenced in Table 5. The labels on the right side give the significance (p-value, T-test) of the difference between the means of the two groups. Each row corresponds to a single CpG and each column to the methylation levels of one sample. CpGs are ordered according to their contribution to the distinction to the differential diagnosis between the two carcinomas with increasing contribution from top to bottom. Black indicates total methylation at a given CpG position, white represents no methylation at the particular position, with degrees of methylation represented in grey, from light (low proportion of methylation) to dark (high proportion of methylation).

SEQ ID NO: 1 to SEQ ID NO: 58 represent 5′ and/or regulatory regions of the genomic DNA of genes MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1. These sequences are derived from Genbank and will be taken to include all minor variations of the sequence material which are currently unforeseen, for example, but not limited to, minor deletions and SNPs.

SEQ ID NO: 76 to SEQ ID NO: 307 exhibit the pretreated sequence of DNA derived from genes MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1. These sequences will be taken to include all minor variations of the sequence material which are currently unforeseen, for example, but not limited to, minor deletions and SNPs.

SEQ ID NO: 308 to SEQ ID NO:427 exhibit the sequence of primer oligonucleotides for the amplification of pretreated DNA according to SEQ ID NO: 76 to SEQ ID NO:307.

SEQ ID NO: 428 to SEQ ID NO: 917 exhibit the sequence of oligomers which are useful for the analysis of CpG positions within genomic DNA according to SEQ ID NO: 1 to SEQ ID NO: 58.

SEQ ID NO: 884 to SEQ ID NO: 917 exhibit the sequence of oligomers which are useful for the analysis of CpG positions within genomic DNA according to SEQ ID NO: 1 to SEQ ID NO: 58.

### EXAMPLE

### Examples 1 and 2

### Digital Phenotype

In the following examples, multiplex PCR was carried out on samples from patients with adenocarcinoma or squamous cell carcinoma. Multiplex PCR was also carried out upon normal tissue adjacent to the carcinoma. Each sample was treated in the manner described below in Example 1 in order to deduce the methylation status of CpG positions, the CpG methylation information for each sample was collated and then used in an analysis, as detailed in Example 2. An alternative method for the analysis of CpG methylation status is further described in Example 3.

### Example 1

In the first step the genomic DNA was isolated from the cell samples using the Wizzard kit from (Promega).

The isolated genomic DNA from the samples are treated using a bisulfite solution (hydrogen sulfite, disulfite). The treatment is such that all non methylated cytosines within the sample are converted to thymidine, conversely 5-methylated cytosines within the sample remain un-modified.

The treated nucleic acids were then amplified using multiplex PCRs, amplifying 8 fragments per reaction with Cy5 fluorescently labelled primers. PCR primers used are described in Table 1. PCR conditions were as follows.

Reaction Solution:

10 ng bisulfite treated DNA
3.5 mM MgCl₂
400 μM dNTPs
2 pmol each primer
1 U Hot Start Taq (Qiagen)

Forty cycles were carried out as follows. Denaturation at 95° C. for 15 min, followed by annealing at 55° C. for 45 sec., primer elongation at 65° C. for 2 min. A final elongation at 65° C. was carried out for 10 min.

All PCR products from each individual sample were then hybridised to glass slides carrying a pair of immobilised oligonucleotides for each CpG position under analysis. Each of these detection oligonucleotides was designed to hybridise to the bisulphite converted sequence around one CpG site which was either originally unmethylated (TG) or methylated (CG). See Table 2 for further details of all hybridisation oligonucleotides used (both informative and non-informative). Hybridisation conditions were selected to allow the detection of the single nucleotide differences between the TG and CG variants.

5 μl volume of each multiplex PCR product was diluted in 10× Ssarc buffer (10× Ssarc: 230 ml 20×SSC, 180 ml sodium lauroyl sarcosinate solution 20% , dilute to 1000 ml with dH2O). The reaction mixture was then hybridised to the detection oligonucleotides as follows. Denaturation at 95° C., cooling down to 10° C., hybridisation at 42° C. overnight followed by washing with 10× Ssarc and dH2O at 42° C.

Fluorescent signals from each hybridised oligonucleotide were detected using genepix scanner and software. Ratios for the two signals (from the CG oligonucleotide and the TG oligonucleotide used to analyse each CpG position) were calculated based on comparison of intensity of the fluorescent signals.

### Example 2

The data obtained according to Example 1 is then sorted into a ranked matrix (as shown in FIGS. 1 to 3) according to CpG methylation differences between the two classes of tissues, using an algorithm. The most significant CpG positions are at the bottom of the matrix with significance decreasing towards the top. Black indicates total methylation at a given CpG position, white represents no methylation at the particular position, with degrees of methylation represented in grey, from light (low proportion of methylation) to dark (high proportion of methylation). Each row represents one specific CpG position within a gene and each column shows the methylation profile for the different CpGs for one sample. On the left side a CpG and gene identifier is shown this may be cross referenced with the accompanying table (Tables 3 to 5) in order to ascertain the gene in question and the detection oligomer used. On the right side p values for the individual CpG positions are shown. The p values are the probabilities that the observed distribution occurred by chance in the data set.

For selected distinctions, we trained a learning algorithm (support vector machine, SVM. The SVM (as discussed by F. Model, P. Adorjan, A. Olek, C. Piepenbrock, Feature selection for DNA methylation based cancer classification. Bioinformatics. 2001 June; 17 Suppl 1:S157-64) constructs an optimal discriminant between two classes of given training samples. In this case each sample is described by the methylation patterns (CG/TG ratios) at the investigated CpG sites. The SVM was trained on a subset of samples of each class, which were presented with the diagnosis attached. Independent test samples, which were not shown to the SVM before were then presented to evaluate, if the diagnosis can be predicted correctly based on the predictor created in the training round. This procedure was repeated several times using different partitions of the samples, a method called crossvalidation. Please note that all rounds are performed without using any knowledge obtained in the previous runs. The number of correct classifications was averaged over all runs, which gives a good estimate of our test accuracy (percent of correct classified samples over all rounds).

Adenocarcinoma Compared to Adjacent Tissue (FIG. 1)

FIG. 1 shows the differentiation of Adenocarcinoma tissue from adjacent tissue using informative CpG positions from 4 genes. Informative CpG positions are further described in Table 3. P values are obtained using the Wilcoxon test.

Squamous Cell Carcinoma Compared to Adjacent Tissue (FIG. 2)

FIG. 2 shows the differentiation of squamous cell carcinoma tissue from adjacent tissue using informative CpG positions from 9 genes. Informative CpG positions are further described in Table 4. P values are obtained using the Wilcoxon test.

Squamous Cell Carcinoma Compared to Adenocarcinoma (FIG. 3)

FIG. 3 shows the differentiation of squamous cell carcinoma from adenocarcinoma. Discrimination between the two classes of carcinomas was possible using CpG positions within two genes. Informative CpG positions are further described in Table 5. P values are obtained using the Wilcoxon test.

### Example 3

### Identification of the Methylation Status of a CpG Site within the Gene RARB

A fragment of the gene RARB was PCR amplified using primers TTCGGATTTTACCATTT (SEQ ID NO: ) and CCTCCCCTGCTCATTTT (SEQ ID NO: ). The resultant fragment (531 bp in length) contained an informative CpG at position 198. The amplificate DNA was digested with the restriction endonuclease AvaI, recognition site CYCGRG. Hydrolysis by said endonuclease is blocked by methylation of the CpG at position 198 of the amplificate. The digest was used as a control.

Genomic DNA was isolated from sample using the DNA wizard DNA isolation kit (Promega). Each sample was digested using AvaI according to manufacturer's recommendations (New England Biolabs).

10 ng of each genomic digest was then amplified using PCR primers TTCGGACCTTTTACCATTT (SEQ ID NO: ) and CCTCCCCTGCTCATTTT (SEQ ID NO: ). The PCR reactions were performed using a thermocycler (Eppendorf GmbH) using 10 ng of DNA, 6 pmole of each primer, 200 μM of each dNTP, 1.5 mM MgCl2 and 1 U of HotstartTaq (Qiagen AG). The other conditions were as recommended by the Taq polymerase manufacturer. Using the above mentioned primers, gene fragments were amplified by PCR performing a first denaturation step for 14 min at 96° C., followed by 30-45 cycles (step 2: 60 sec at 96° C., step 3: 45 sec at 52° C. , step 4: 75 sec at 72° C.) and a subsequent final elongation of 10 min at 72° C. The presence of PCR products was analysed by agarose gel electrophoresis.

PCR products were detectable with AvaI hydrolysed DNA isolated wherein the CpG position in question was up-methylated, when step 2 to step 4 of the cycle program were repeated 34, 37, 39, 42 and 45 fold. In contrast PCR products were only detectable with AvaI hydrolysed DNA isolated from down-methylated DNA (and control DNA) when step 2 to step 4 of the cycle program were repeated 42- and 45-fold. These results were incorporated into a CpG methylation matrix analysis as described in Example 2.

### Tables

TABLE 1
PCR primers and products

| | | | Primer | |
|---|---|---|---|---|
| No: | Gene | Primer: | type: | Size: |
| 1 | MDR1 | TAAGTATGTTGAAGAAAGATTATTGTAG | start | 633 |
| | (SEQ ID NO: 1) | (SEQ ID NO: 308) | | |
| | | TAAAAACTATCCCATAATAACTCCCAAC | stop | |
| | | (SEQ ID NO: 309) | | |
| 2 | APOC2 | ATGAGTAGAAGAGGTGATAT | start | 533 |
| | (SEQ ID NO: 2) | (SEQ ID NO: 310) | | |
| | | CCCTAAATCCCTTTCTTACC | stop | |
| | | (SEQ ID NO: 311) | | |
| 3 | CACNA1G | GGGATTTAAGAGAAATTGAGGTA | start | 707 |
| | (SEQ ID NO: 3) | (SEQ ID NO: 312) | | |
| | | AAACCCCAAACATCCTTTAT | stop | |
| | | (SEQ ID NO: 313) | | |
| 4 | EGR4 | AGGGGGATTGAGTGTTAAGT | start | 293 |
| | (SEQ ID NO: 4) | (SEQ ID NO: 315) | | |
| | | CCCAAACATAAACACAAAAT | stop | |
| | | (SEQ ID NO: 314) | | |
| 5 | AR | GTAGTAGTAGTAGTAAGAGA | start | 460 |
| | (SEQ ID NO: 5) | (SEQ ID NO: 316) | | |
| | | ACCCCCTAAATAATTATCCT | stop | |
| | | (SEQ ID NO: 317) | | |
| 6 | RB1 | TTTAAGTTTGTTTTTGTTTTGGT | start | 718 |
| | (SEQ ID NO: 6) | (SEQ ID NO: 318) | | |
| | | TCCTACTCTAAATCCTCCTCAA | stop | |
| | | (SEQ ID NO: 319) | | |
| 7 | GPIb beta | GGTGATAGGAGAATAATGTTGG | start | 379 |
| | (SEQ ID NO: 7) | (SEQ ID NO: 320) | | |
| | | TCCTCCCAACTACAACCAAAC | stop | |
| | | (SEQ ID NO: 321) | | |
| 8 | MYOD1 | ATTAGGGGTATAGAGGAGTATTGA | start | 883 |
| | (SEQ ID NO: 8) | (SEQ ID NO: 322) | | |
| | | CTTACAAACCCACAATAAACAA | stop | |
| | | (SEQ ID NO: 323) | | |
| 9 | WT1 | AAAGGGAAATTAAGTGTTGT | start | 747 |
| | (SEQ ID NO: 9) | (SEQ ID NO: 325) | | |
| | | TAACTACCCTCAACTTCCC | stop | |
| | | (SEQ ID NO: 324) | | |
| 10 | HLA-F | TTGTTGTTTTTAGGGGTTTTGG | start | 946 |
| | (SEQ ID NO: 10) | (SEQ ID NO: 326) | | |
| | | TCCTTCCCATTCTCCAAATATC | stop | |
| | | (SEQ ID NO: 327) | | |
| 11 | ELK1 | AAGTGTTTTAGTTTTTAATGGGTA | start | 966 |
| | (SEQ ID NO: 11) | (SEQ ID NO: 328) | | |
| | | CAAACCCAAAACTCACCTAT | stop | |
| | | (SEQ ID NO: 329) | | |
| 12 | APC | AGGAAGTATTGAAGATGAAGTTATG | start | |
| | (SEQ ID NO: 12) | (SEQ ID NO: 330) | | |
| | | TTCCAATAAAACAATAAACTC | stop | |
| | | (SEQ ID NO: 331) | | |
| 13 | ARHI | GTGAGTTTTTGGGGTGTTTA | start | 442 |
| | (SEQ ID NO: 13) | (SEQ ID NO: 332) | | |
| | | TCAATCTTACTTTCACACTACATAA | stop | |
| | | (SEQ ID NO: 333) | | |
| 14 | BCL2 | GTATTTTATGTTAAGGGGGAAA | start | 640 |
| | SEQ ID NO: 14) | SEQ ID NO: 334) | | |
| | | AAAAACCACAATCCTCCC | stop | |
| | | SEQ ID NO: 335) | | |
| 15 | BRCA1 | TGGATGGGAATTGTAGTTTT | start | 537 |
| | (SEQ ID NO: 15) | (SEQ ID NO: 336) | | |
| | | TTAACCACCCAATCTACCC | stop | |
| | | (SEQ ID NO: 337) | | |
| 16 | CACLA | GTTTTGGAAGTATGAGGGTG | start | 614 |
| | (SEQ ID NO: 16) | (SEQ ID NO: 338) | | |
| | | CCAAATTCTAAACCAATTTCC | stop | |
| | | (SEQ ID NO: 339) | | |
| 17 | CCND2 | TTTTGGTATGTAGGTTGGATG | start | 426 |
| | (SEQ ID NO: 17) | (SEQ ID NO: 340) | | |
| | | CCTAACCTCCTTCCTTTAACT | stop | |
| | | (SEQ ID NO: 341) | | |
| 18 | CDH1 | CAAATAAACCCTCAACCAATC | start | 474 |
| | (SEQ ID NO: 18) | (SEQ ID NO: 342) | | |
| | | TGGAGGGGGTAGGAAAGT | stop | |
| | | (SEQ ID NO: 343) | | |
| 19 | CDKN1B | GTGGGGAGGTAGTTGAAGA | start | 478 |
| | (SEQ ID NO: 19) | (SEQ ID NO: 344) | | |
| | | ATACACCCCTAACCCAAAAT | stop | |
| | | (SEQ ID NO: 345) | | |
| 20 | CDKN2a | TTGAAAATTAAGGGTTGAGG | start | 598 |
| | (SEQ ID NO: 20) | (SEQ ID NO: 346) | | |
| | | CACCCTCTAATAACCAACCA | stop | |
| | | (SEQ ID NO: 347) | | |
| 21 | CDKN2a | GGGGTTGGTTGGTTATTAGA | start | 256 |
| | (SEQ ID NO: 20) | (SEQ ID NO: 348) | | |
| | | AACCCTCTACCCACCTAAAT | stop | |
| | | (SEQ ID NO: 349) | | |
| 22 | CDKN2B | GGTTGGTTGAAGGAATAGAAAT | start | 708 |
| | (SEQ ID NO: 21) | (SEQ ID NO: 350) | | |
| | | CCCACTAAACATACCCTTATTC | stop | |
| | | (SEQ ID NO: 351) | | |
| 23 | CD44 | GAAAGGAGAGGTTAAAGGTTG | start | 696 |
| | (SEQ ID NO: 22) | (SEQ ID NO: 352) | | |
| | | AACTCACTTAACTCCAATCCC | stop | |
| | | (SEQ ID NO: 353) | | |
| 24 | CSPG2 | GGATAGGAGTTGGGATTAAGAT | start | 414 |
| | (SEQ ID NO: 23) | (SEQ ID NO: 354) | | |
| | | AAATCTTTTTCAACACCAAAAT | stop | |
| | | (SEQ ID NO: 355) | | |
| 25 | DAPK1 | AACCCTTTCTTCAAATTACAAA | start | 348 |
| | (SEQ ID NO: 24) | (SEQ ID NO: 356) | | |
| | | TGATTGGGTTTTAGGGAAATA | stop | |
| | | (SEQ ID NO: 357) | | |
| 26 | GGT1 | GTGAAGGGTGTGAGTTGTTTA | start | 562 |
| | (SEQ ID NO: 25) | (SEQ ID NO: 358) | | |
| | | CACAATCAATTTCCCACAA | stop | |
| | | (SEQ ID NO: 359) | | |
| 27 | GSTP1 | ATTTGGGAAAGAGGGAAAG | start | 300 |
| | (SEQ ID NO: 26) | (SEQ ID NO: 360) | | |
| | | TAAAAACTCTAAACCCCATCC | stop | |
| | | (SEQ ID NO: 361) | | |
| 28 | HIC-1 | TGGGTTGGAGAAGAAGTTTA | start | 280 |
| | (SEQ ID NO: 27) | (SEQ ID NO: 362) | | |
| | | TCATATTTCCAAAAACACACC | stop | |
| | | (SEQ ID NO: 363) | | |
| 29 | LAP18 | GAGTTTGTATTTAAGTTGAGTGGTT | start | 334 |
| | (SEQ ID NO: 28) | (SEQ ID NO: 364) | | |
| | | AACAAAACAATACCCCTTCTAA | stop | |
| | | (SEQ ID NO: 365) | | |
| 30 | LKB1 | TAAAAGAAGGATTTTTGATTGG | start | 528 |
| | (SEQ ID NO: 29) | (SEQ ID NO: 367) | | |
| | | CATCTTATTTACCTCCCTCCC | stop | |
| | | (SEQ ID NO: 366) | | |
| 31 | LOC51147 | ATTAGGGATGAGAGGATTTGTA | start | 212 |
| | (SEQ ID NO: 30) | (SEQ ID NO: 368) | | |
| | | TCTTCCTAACCATACACACTAACC | stop | |
| | | (SEQ ID NO: 369) | | |
| 32 | MGMT | AAGGTTTTAGGGAAGAGTGTTT | start | 636 |
| | (SEQ ID NO: 31) | (SEQ ID NO: 370) | | |
| | | ACCTTTTCCTATCACAAAAATAA | stop | |
| | | (SEQ ID NO: 371) | | |
| 33 | MLH1 | TAAGGGGAGAGGAGGAGTTT | start | 545 |
| | (SEQ ID NO: 32) | (SEQ ID NO: 372) | | |
| | | ACCAATTCTCAATCATCTCTTT | stop | |
| | | (SEQ ID NO: 373) | | |
| 34 | MNCA9 | GGGAAGTAGGTTAGGGTTAGTT | start | |
| | (SEQ ID NO: 33) | (SEQ ID NO: 374) | | |
| | | AAATCCTCCTCTCCAAATAAAT | stop | |
| | | (SEQ ID NO: 375) | | |
| 35 | MYC | AGAGGGAGTAAAAGAAAATGGT | start | 712 |
| | (SEQ ID NO: 34) | (SEQ ID NO: 376) | | |
| | | CCAAATAAACAAAATAACCTCC | stop | |
| | | (SEQ ID NO: 377) | | |
| 36 | N33 | TTTTAGATTGAGGTTTTAGGGT | start | 497 |
| | (SEQ ID NO: 35) | (SEQ ID NO: 378) | | |
| | | ATCCATTCTACCTCCTTTTTCT | stop | |
| | | (SEQ ID NO: 379) | | |
| 37 | PAX6 | GGAGGGGAGAGGGTTATG | start | 374 |
| | (SEQ ID NO: 36) | (SEQ ID NO: 380) | | |
| | | TACTATACACACCCCAAAACAA | stop | |
| | | (SEQ ID NO: 381) | | |
| 38 | PGR | TTTTGGGAATGGGTTGTAT | start | 369 |
| | (SEQ ID NO: 37) | (SEQ ID NO: 382) | | |
| | | CTACCCTTAACCTCCATCCTA | stop | |
| | | (SEQ ID NO: 383) | | |
| 39 | PTEN | TTTTAGGTAGTTATATTGGGTATGTT | start | 346 |
| | (SEQ ID NO: 38) | (SEQ ID NO: 384) | | |
| | | TCAACTCTCAAACTTCCATCA | stop | |
| | | (SEQ ID NO: 385) | | |
| 40 | RARB | TTGTTGGGAGTTTTTAAGTTTT | start | 353 |
| | (SEQ ID NO: 39) | (SEQ ID NO: 386) | | |
| | | CAAATTCTCCTTCCAAATAAAT | stop | |
| | | (SEQ ID NO: 387) | | |
| 41 | SFN | GAAGAGAGGAGAGGGAGGTA | start | 489 |
| | (SEQ ID NO: 40) | (SEQ ID NO: 389) | | |
| | | CTATCCAACAAACCCAACA | stop | |
| | | (SEQ ID NO: 388) | | |
| 42 | S100A2 | GTTTTTAAGTTGGAGAAGAGGA | start | 460 |
| | (SEQ ID NO: 41) | (SEQ ID NO: 390) | | |
| | | ACCTATAAATCACAACCCACTC | stop | |
| | | (SEQ ID NO: 391) | | |
| 43 | TFF1 | GGTTTTGGTGTTTATGTTGGT | start | |
| | (SEQ ID NO: 42) | (SEQ ID NO: 393) | | |
| | | AAATCCCTACAAAAATATCTAAAA | stop | |
| | | (SEQ ID NO: 392) | | |
| 44 | TGFBR2 | GTAATTTGAAGAAAGTTGAGGG | start | 296 |
| | (SEQ ID NO: 43) | (SEQ ID NO: 394) | | |
| | | CCAACAACTAAACAAAACCTCT | stop | |
| | | (SEQ ID NO: 395) | | |
| 45 | TIMP3 | TGAGAAAATTGTTGTTTGAAGT | start | 306 |
| | (SEQ ID NO: 44) | (SEQ ID NO: 396) | | |
| | | CAAAATACCCTAAAAACCACTC | stop | |
| | | (SEQ ID NO: 397) | | |
| 46 | VHL | TGTAAAATGAATAAAGTTAATGAGTG | start | 362 |
| | (SEQ ID NO: 45) | (SEQ ID NO: 398) | | |
| | | TCCTAAATTCAAATAATCCTCCT | stop | |
| | | (SEQ ID NO: 399) | | |
| 47 | CDKN1C | GGGGAGGTAGATATTTGGATAA | start | 300 |
| | (SEQ ID NO: 46) | (SEQ ID NO: 400) | | |
| | | AACTACACCATTTATATTCCCAC | stop | |
| | | (SEQ ID NO: 401) | | |
| 48 | CAV1 | GTTAGTATGTTTGGGGGTAAAT | start | 435 |
| | (SEQ ID NO: 47) | (SEQ ID NO: 403) | | |
| | | ATAAATAACACCTTCCACCCTA | stop | |
| | | (SEQ ID NO: 402) | | |
| 49 | CDH13 | TTTGTATTAGGTTGGAAGTGGT | start | 286 |
| | (SEQ ID NO: 48) | (SEQ ID NO: 404) | | |
| | | CCCAAATAAATCAACAACAACA | stop | |
| | | (SEQ ID NO: 405) | | |
| 50 | NDRG1 | GGTTTTGGGTTTAGTGGTAAAT | start | 416 |
| | (SEQ ID NO: 49) | (SEQ ID NO: 407) | | |
| | | AACTTTCATAACTCACCCTTTC | stop | |
| | | (SEQ ID NO: 406) | | |
| 51 | PTGS2 | GATTTTTGGAGAGGAAGTTAAG | start | 381 |
| | (SEQ ID NO: 50) | (SEQ ID NO: 409) | | |
| | | AAAACTAAAAACCAAACCCATA | stop | |
| | | (SEQ ID NO: 408) | | |
| 52 | THBS1 | TGGGGTTAGTTTAGGATAGG | start | 398 |
| | (SEQ ID NO: 51) | (SEQ ID NO: 410) | | |
| | | CTTAAAAACACTAAAACTTCTCAAA | stop | |
| | | (SEQ ID NO: 411) | | |
| 53 | TMEFF2 | TTGTTTGGGTTAATAAATGGA | start | 295 |
| | (SEQ ID NO: 52) | (SEQ ID NO: 412) | | |
| | | CTTCTCTCTTCTCCCCTCTC | stop | |
| | | (SEQ ID NO: 413) | | |
| 54 | TMEFF2 | TGTTGGTTGTTGTTGTTGTT | start | 319 |
| | (SEQ ID NO: 52) | (SEQ ID NO: 414) | | |
| | | CTTTCTACCCATCCCAAAA | stop | |
| | | (SEQ ID NO: 415) | | |
| 55 | PLAU | TATTATAGGAGGATTGAGGAGG | start | 499 |
| | (SEQ ID NO: 53) | (SEQ ID NO: 416) | | |
| | | CCCATAAAATCATACCACTTCT | stop | |
| | | (SEQ ID NO: 417) | | |
| 56 | DNMT1 | TCCCCATCACACCTAAAA | start | 210 |
| | (SEQ ID NO: 54) | (SEQ ID NO: 418) | | |
| | | GGGAGGAGGGGATGTATT | stop | |
| | | (SEQ ID NO: 419) | | |
| 57 | ESR1 | AGGGGGAATTAAATAGAAAGAG | start | 662 |
| | (SEQ ID NO: 55) | (SEQ ID NO: 420) | | |
| | | CAATAAAACCATCCCAAATACT | stop | |
| | | (SEQ ID NO: 421) | | |
| 58 | APAF1 | AGATATGTTTGGAGATTTTAGGA | start | 674 |
| | (SEQ ID NO: 56) | (SEQ ID NO: 422) | | |
| | | AACTCCCCACCTCTAATTCTAT | stop | |
| | | (SEQ ID NO: 423) | | |
| 59 | HOXA5 | AAACCCCAAACAACCTCTAT | start | 392 |
| | (SEQ ID NO: 57) | (SEQ ID NO: 425) | | |
| | | GAAGGGGGAAAGTTATTTAGTTA | stop | |
| | | (SEQ ID NO: 424) | | |
| 60 | RASSF1 | ACCTCTCTACAAATTACAAATTCA | start | 347 |
| | (SEQ ID NO: 58) | (SEQ ID NO: 426) | | |
| | | AGTTTGGGTTAGTTTGGGTT | stop | |
| | | (SEQ ID NO: 427) | | |

TABLE 2
Hybridisation oligonucleotides

| | | |
|---|---|---|
| No: | Gene | Oligo: |

| | | |
|---|---|---|
| 1 | MDR1 | TTGGTGGTCGTTTTAAGG |
| | (SEQ ID NO: 1) | (SEQ ID NO: 428) |
| 2 | MDR1 | TTGGTGGTTGTTTTAAGG |
| | (SEQ ID NO: 1) | (SEQ ID NO: 429) |
| 3 | MDR1 | TTGAAAGACGTGTTTATA |
| | (SEQ ID NO: 1) | (SEQ ID NO: 430) |
| 4 | MDR1 | TTGAAAGATGTGTTTATA |
| | (SEQ ID NO: 1) | (SEQ ID NO: 431) |
| 5 | MFR1 | AGGTGTAACGGAAGTTAG |
| | (SEQ ID NO: 1) | (SEQ ID NO: 432) |
| 6 | MFR1 | AGGTGTAATGGAAGTTAG |
| | (SEQ ID NO: 1) | (SEQ ID NO: 433) |
| 7 | MFR1 | TAGTTTTTCGAGGAATTA |
| | (SEQ ID NO: 1) | (SEQ ID NO: 434) |
| 8 | MDR1 | TAGTTTTTTGAGGAATTA |
| | (SEQ ID NO: 1) | (SEQ ID NO: 435) |
| 9 | APOC2 | TTTTAAGGCGTGTTAGTT |
| | (SEQ ID NO: 2) | (SEQ ID NO: 436) |
| 10 | APOC2 | TTTTAAGGTGTGTTAGTT |
| | (SEQ ID NO: 2) | (SEQ ID NO: 437) |
| 11 | APOC2 | TTTTGTGACGTGATTTTG |
| | (SEQ ID NO: 2) | (SEQ ID NO: 438) |
| 12 | APOC2 | TTTTGTGATGTGATTTTG |
| | (SEQ ID NO: 2) | (SEQ ID NO: 439) |
| 13 | APOC2 | TTGGGGGACGTTATTGTT |
| | (SEQ ID NO: 2) | (SEQ ID NO: 440) |
| 14 | APOC2 | TTGGGGGATGTTATTGTT |
| | (SEQ ID NO: 2) | (SEQ ID NO: 441) |
| 15 | APOC2 | TGGGTTTGCGGAGAATGG |
| | (SEQ ID NO: 2) | (SEQ ID NO: 442) |
| 16 | APOC2 | TGGGTTTGTGGAGAATGG |
| | (SEQ ID NO: 2) | (SEQ ID NO: 443) |
| 17 | CACNA1G | GTTTAGCGCGATTTGTTT |
| | (SEQ ID NO: 3) | (SEQ ID NO: 444) |
| 18 | CACNA1G | GTTTAGTGTGATTTGTTT |
| | (SEQ ID NO: 3) | (SEQ ID NO: 445) |
| 19 | CACNA1G | TTTAGGAGCGTTAATGTG |
| | (SEQ ID NO: 3) | (SEQ ID NO: 446) |
| 20 | CACNA1G | TTTAGGAGTGTTAATGTG |
| | (SEQ ID NO: 3) | (SEQ ID NO: 447) |
| 21 | CACNA1G | TAGGGTTACGAGGTTAGG |
| | (SEQ ID NO: 3) | (SEQ ID NO: 448) |
| 22 | CACNA1G | TAGGGTTATGAGGTTAGG |
| | (SEQ ID NO: 3) | (SEQ ID NO: 449) |
| 23 | CACNA1G | TTTAGGTTCGTTTAGAGT |
| | (SEQ ID NO: 3) | (SEQ ID NO: 450) |
| 24 | CACNA1G | TTTAGGTTTGTTTAGAGT |
| | (SEQ ID NO: 3) | (SEQ ID NO: 451) |
| 25 | CACNA1G | TTAGGGGTCGTGGATAAA |
| | (SEQ ID NO: 3) | (SEQ ID NO: 452) |
| 26 | CACNA1G | TTAGGGGTTGTGGATAAA |
| | (SEQ ID NO: 3) | (SEQ ID NO: 453) |
| 27 | EGR4 | GGTGGGAAGCGTATTTAT |
| | (SEQ ID NO: 4) | (SEQ ID NO: 454) |
| 28 | EGR4 | GGTGGGAAGTGTATTTAT |
| | (SEQ ID NO: 4) | (SEQ ID NO: 455) |
| 29 | EGR4 | AATAATAACGTTATAGTT |
| | (SEQ ID NO: 4) | (SEQ ID NO: 456) |
| 30 | EGR4 | AATAATAATGTTATAGTT |
| | (SEQ ID NO: 4) | (SEQ ID NO: 457) |
| 31 | EGR4 | TTATAGTTCGAGTTTTTT |
| | (SEQ ID NO: 4) | (SEQ ID NO: 458) |
| 32 | EGR4 | TTATAGTTTGAGTTTTTT |
| | (SEQ ID NO: 4) | (SEQ ID NO: 459) |
| 33 | EGR4 | GGAGTTTTCGGTATATAT |
| | (SEQ ID NO: 4) | (SEQ ID NO: 460) |
| 34 | EGR4 | GGAGTTTTTGGTATATAT |
| | (SEQ ID NO: 4) | (SEQ ID NO: 461) |
| 35 | AR | TGTTATTTCGAGAGAGGT |
| | (SEQ ID NO: 5) | (SEQ ID NO: 462) |
| 36 | AR | TGTTATTTTGAGAGAGGT |
| | (SEQ ID NO: 5) | (SEQ ID NO: 463) |
| 37 | AR | AGAGGTTGCGTTTTAGAG |
| | (SEQ ID NO: 5) | (SEQ ID NO: 464) |
| 38 | AR | AGAGGTTGTGTTTTAGAG |
| | (SEQ ID NO: 5) | (SEQ ID NO: 465) |
| 39 | AR | GTAGTATTCGAAGGTAGT |
| | (SEQ ID NO: 5) | (SEQ ID NO: 466) |
| 40 | AR | GTAGTATTTGAAGGTAGT |
| | (SEQ ID NO: 5) | (SEQ ID NO: 467) |
| 41 | AR | GGAGGTTTCGGGGGTTTT |
| | (SEQ ID NO: 5) | (SEQ ID NO: 468) |
| 42 | AR | GGAGGTTTTGGGGGTTTT |
| | (SEQ ID NO: 5) | (SEQ ID NO: 469) |
| 43 | RB1 | TTAGATTTCGGGATAGGG |
| | (SEQ ID NO: 6) | (SEQ ID NO: 470) |
| 44 | RB1 | TTAGATTTTGGGATAGGG |
| | (SEQ ID NO: 6) | (SEQ ID NO: 471) |
| 45 | RB1 | TATAGTTTCGTTAAGTGT |
| | (SEQ ID NO: 6) | (SEQ ID NO: 472) |
| 46 | RB1 | TATAGTTTTGTTAAGTGT |
| | (SEQ ID NO: 6) | (SEQ ID NO: 473) |
| 47 | RB1 | GTGTATTTCGGTTTGGAG |
| | (SEQ ID NO: 6) | (SEQ ID NO: 474) |
| 48 | RB1 | GTGTATTTTGGTTTGGAG |
| | (SEQ ID NO: 6) | (SEQ ID NO: 475) |
| 49 | RB1 | TTGGAAGGCGTTTGGATT |
| | (SEQ ID NO: 6) | (SEQ ID NO: 476) |
| 50 | RB1 | TTGGAAGGTGTTTGGATT |
| | (SEQ ID NO: 6) | (SEQ ID NO: 477) |
| 51 | GPIb beta | TTTGAGAGCGGGTGGGAG |
| | (SEQ ID NO: 7) | (SEQ ID NO: 898) |
| 52 | GPIb beta | TTTGAGAGTGGGTGGGAG |
| | (SEQ ID NO: 7) | (SEQ ID NO: 899) |
| 53 | GPIb beta | GTGGGAGCGGAAGTTTGA |
| | (SEQ ID NO: 7) | (SEQ ID NO: 904) |
| 54 | GPIb beta | GTGGGAGTGGAAGTTTGA |
| | (SEQ ID NO: 7) | (SEQ ID NO: 905) |
| 55 | GPIb beta | GGTTAGGTCGTAGTATTG |
| | (SEQ ID NO: 7) | (SEQ ID NO: 478) |
| 56 | GPIb beta | GGTTAGGTTGTAGTATTG |
| | (SEQ ID NO: 7) | (SEQ ID NO: 479) |
| 57 | GPIb beta | ATGGGTTTCGGTGAGTTT |
| | (SEQ ID NO: 7) | (SEQ ID NO: 480) |
| 58 | GPIb beta | ATGGGTTTTGGTGAGTTT |
| | (SEQ ID NO: 7) | (SEQ ID NO: 481) |
| 59 | MYOD1 | ATAGTAGTCGGGTGTTGG |
| | (SEQ ID NO: 8) | (SEQ ID NO: 482) |
| 60 | MYOD1 | ATAGTAGTTGGGTGTTGG |
| | (SEQ ID NO: 8) | (SEQ ID NO: 483) |
| 61 | MYOD1 | GTGTTAGTCGTTTAGGGT |
| | (SEQ ID NO: 8) | (SEQ ID NO: 484) |
| 62 | MYOD1 | GTGTTAGTTGTTTAGGGT |
| | (SEQ ID NO: 8) | (SEQ ID NO: 486) |
| 63 | MYOD1 | TAGTTGTTTGTTTGGGTT |
| | (SEQ ID NO: 8) | (SEQ ID NO: 487) |
| 64 | MYOD1 | TAGTTGTTTGTTTGGGTT |
| | (SEQ ID NO: 8) | (SEQ ID NO: 487) |
| 65 | MYOD1 | GGTTATTACGGATAAATA |
| | (SEQ ID NO: 8) | (SEQ ID NO: 488) |
| 66 | MYOD1 | GGTTATTATGGATAAATA |
| | (SEQ ID NO: 8) | (SEQ ID NO: 489) |
| 67 | WT1 | ATTTTGTTCGGATTTATT |
| | (SEQ ID NO: 9) | (SEQ ID NO: 490) |
| 68 | WT1 | ATTTTGTTTGGATTTATT |
| | (SEQ ID NO: 9) | (SEQ ID NO: 491) |
| 69 | WT1 | TATTTGAACGGATTTTTT |
| | (SEQ ID NO: 9) | (SEQ ID NO: 492) |
| 70 | WT1 | TATTTGAATGGATTTTTT |
| | (SEQ ID NO: 9) | (SEQ ID NO: 493) |
| 71 | WT1 | TGTTATATCGGTTAGTTG |
| | (SEQ ID NO: 9) | (SEQ ID NO: 494) |
| 72 | WT1 | TGTTATATTGGTTAGTTG |
| | (SEQ ID NO: 9) | (SEQ ID NO: 495) |
| 73 | WT1 | TGTTTGGTCGGGTTTGGG |
| | (SEQ ID NO: 9) | (SEQ ID NO: 496) |
| 74 | WT1 | TGTTTGGTTGGGTTTGGG |
| | (SEQ ID NO: 9) | (SEQ ID NO: 497) |
| 75 | HLA-F | TATTTGGGCGGGTGAGTG |
| | (SEQ ID NO: 10) | (SEQ ID NO: 894) |
| 76 | HLA-F | TATTTGGGTGGGTGAGTG |
| | (SEQ ID NO: 10) | (SEQ ID NO: 895) |
| 77 | HLA-F | AAAATTTTCGCGGGTTGG |
| | (SEQ ID NO: 10) | (SEQ ID NO: 498) |
| 78 | HLA-F | AAAATTTTTGTGGGTTGG |
| | (SEQ ID NO: 10) | (SEQ ID NO: 499) |
| 79 | HLA-F | GAGAGAAACGGTTTTTGT |
| | (SEQ ID NO: 10) | (SEQ ID NO: 500) |
| 80 | HLA-F | GAGAGAAATGGTTTTTGT |
| | (SEQ ID NO: 10) | (SEQ ID NO: 501) |
| 81 | HLA-F | GAGTTGTTTCGTAGATAT |
| | (SEQ ID NO: 10) | (SEQ ID NO: 502) |
| 82 | HLA-F | GAGTTGTTTTGTAGATAT |
| | (SEQ ID NO: 10) | (SEQ ID NO: 503) |
| 83 | ELK1 | TTTGTTTTCGTTGAGTAG |
| | (SEQ ID NO: 11) | (SEQ ID NO: 504) |
| 84 | ELK1 | TTTGTTTTTGTTGAGTAG |
| | (SEQ ID NO: 11) | (SEQ ID NO: 505) |
| 85 | ELK1 | TTTATTTTCGTTTTTGGG |
| | (SEQ ID NO: 11) | (SEQ ID NO: 506) |
| 86 | ELK1 | TTTATTTTTGTTTTTGGG |
| | (SEQ ID NO: 11) | (SEQ ID NO: 507) |
| 87 | ELK1 | GAAGGGTTCGTTTTTTAA |
| | (SEQ ID NO: 11) | (SEQ ID NO: 508) |
| 88 | ELK1 | GAAGGGTTTGTTTTTTAA |
| | (SEQ ID NO: 11) | (SEQ ID NO: 509) |
| 89 | ELK1 | ATTAATAGCGTTTTGGTT |
| | (SEQ ID NO: 11) | (SEQ ID NO: 510) |
| 90 | ELK1 | ATTAATAGTGTTTTGGTT |
| | (SEQ ID NO: 11) | (SEQ ID NO: 511) |
| 91 | APC | TATTAGAGCGTTTTAAAG |
| | (SEQ ID NO: 12) | (SEQ ID NO: 512) |
| 92 | APC | TATTAGAGTGTTTTAAAG |
| | (SEQ ID NO: 12) | (SEQ ID NO: 513) |
| 93 | APC | GTTTTTTTCGATTTGGGT |
| | (SEQ ID NO: 12) | (SEQ ID NO: 514) |
| 94 | APC | GTTTTTTTTGATTTGGGT |
| | (SEQ ID NO: 12) | (SEQ ID NO: 515) |
| 95 | ARHI | TTGGTTGTCGCGGTAGTT |
| | (SEQ ID NO: 13) | (SEQ ID NO: 516) |
| 96 | ARHI | TTGGTTGTTGTGGTAGTT |
| | (SEQ ID NO: 13) | (SEQ ID NO: 517) |
| 97 | ARHI | TGTTGTTGCGTAGTAGAA |
| | (SEQ ID NO: 13) | (SEQ ID NO: 518) |
| 98 | ARHI | TGTTGTTGTGTAGTAGAA |
| | (SEQ ID NO: 13) | (SEQ ID NO: 519) |
| 99 | ARHI | GAATTATTCGTAGTTTTG |
| | (SEQ ID NO: 13) | (SEQ ID NO: 520) |
| 100 | ARHI | GAATTATTTGTAGTTTTG |
| | (SEQ ID NO: 13) | (SEQ ID NO: 521) |
| 101 | ARHI | TAGAAGAACGAGGTTTGA |
| | (SEQ ID NO: 13) | (SEQ ID NO: 522) |
| 102 | ARHI | TAGAAGAATGAGGTTTGA |
| | (SEQ ID NO: 13) | (SEQ ID NO: 523) |
| 103 | ARHI | TAAGTGTGCGAGTTTAAA |
| | (SEQ ID NO: 13) | (SEQ ID NO: 524) |
| 104 | ARHI | TAAGTGTGTGAGTTTAAA |
| | (SEQ ID NO: 13) | (SEQ ID NO: 525) |
| 105 | BCL2 | AGTGTTTCGCGTGATTGA |
| | (SEQ ID NO: 14) | (SEQ ID NO: 526) |
| 106 | BCL2 | AGTGTTTTGTGTGATTGA |
| | (SEQ ID NO: 14) | (SEQ ID NO: 527) |
| 107 | BCL2 | AGTTGGGGCGAGAGGTGT |
| | (SEQ ID NO: 14) | (SEQ ID NO: 528) |
| 108 | BCL2 | AGTTGGGGTGAGAGGTGT |
| | (SEQ ID NO: 14) | (SEQ ID NO: 529) |
| 109 | BCL2 | TAAGTTGTCGTAGAGGGG |
| | (SEQ ID NO: 14) | (SEQ ID NO: 530) |
| 110 | BCL2 | TAAGTTGTTGTAGAGGGG |
| | (SEQ ID NO: 14) | (SEQ ID NO: 531) |
| 111 | BCL2 | AGGGGTTACGAGTGGGAT |
| | (SEQ ID NO: 14) | (SEQ ID NO: 532) |
| 112 | BCL2 | AGGGGTTATGAGTGGGAT |
| | (SEQ ID NO: 14) | (SEQ ID NO: 533) |
| 113 | BCL2 | AGGATTTCGTCGTTGTAG |
| | (SEQ ID NO: 14) | (SEQ ID NO: 534) |
| 114 | BCL2 | AGGATTTTGTTGTTGTAG |
| | (SEQ ID NO: 14) | (SEQ ID NO: 535) |
| 115 | BRCA1 | TGGATTTTCGTGAGAATT |
| | (SEQ ID NO: 15) | (SEQ ID NO: 536) |
| 116 | BRCA1 | TGGATTTTTGTGAGAATT |
| | (SEQ ID NO: 15) | (SEQ ID NO: 537) |
| 117 | BRCA1 | ATTGTGTTCGTTTTGGTA |
| | (SEQ ID NO: 15) | (SEQ ID NO: 538) |
| 118 | BRCA1 | ATTGTGTTTGTTTTGGTA |
| | (SEQ ID NO: 15) | (SEQ ID NO: 539) |
| 119 | BRCA1 | TATTGTGGCGAAGATTTT |
| | (SEQ ID NO: 15) | (SEQ ID NO: 540) |
| 120 | BRCA1 | TATTGTGGTGAAGATTTT |
| | (SEQ ID NO: 15) | (SEQ ID NO: 541) |
| 121 | BRCA1 | TAATAAGTCGTAATTGGA |
| | (SEQ ID NO: 15) | (SEQ ID NO: 542) |
| 122 | BRCA1 | TAATAAGTTGTAATTGGA |
| | (SEQ ID NO: 15) | (SEQ ID NO: 543) |
| 123 | CALCA | GAGGGTGACGTAATTTAG |
| | (SEQ ID NO: 16) | (SEQ ID NO: 544) |
| 124 | CALCA | GAGGGTGATGTAATTTAG |
| | (SEQ ID NO: 16) | (SEQ ID NO: 545) |
| 125 | CALCA | TGTATTGGCGGAATTTTT |
| | (SEQ ID NO: 16) | (SEQ ID NO: 546) |
| 126 | CALCA | TGTATTGGTGGAATTTTT |
| | (SEQ ID NO: 16) | (SEQ ID NO: 547) |
| 127 | CALCA | ATTTATAGCGGCGGGAAT |
| | (SEQ ID NO: 16) | (SEQ ID NO: 548) |
| 128 | CALCA | ATTTATAGTGGTGGGAAT |
| | (SEQ ID NO: 16) | (SEQ ID NO: 549) |
| 129 | CALCA | TGTTAGTTCGCGATTTAT |
| | (SEQ ID NO: 16) | (SEQ ID NO: 550) |
| 130 | CALCA | TGTTAGTTTGTGATTTAT |
| | (SEQ ID NO: 16) | (SEQ ID NO: 551) |
| 131 | CALCA | GGTTGGATCGGATAGTTT |
| | (SEQ ID NO: 16) | (SEQ ID NO: 552) |
| 132 | CALCA | GGTTGGATTGGATAGTTT |
| | (SEQ ID NO: 16) | (SEQ ID NO: 553) |
| 133 | CCND2 | TTTAATAACGAGAGGGGA |
| | (SEQ ID NO: 17) | (SEQ ID NO: 554) |
| 134 | CCND2 | TTTAATAATGAGAGGGGA |
| | (SEQ ID NO: 17) | (SEQ ID NO: 555) |
| 135 | CCND2 | TTAGTTTGCGTTATCGTT |
| | (SEQ ID NO: 17) | (SEQ ID NO: 556) |
| 136 | CCND2 | TTAGTTTGTGTTATTGTT |
| | (SEQ ID NO: 17) | (SEQ ID NO: 557) |
| 137 | CCND2 | TTTTAGAGCGGAGAAGAG |
| | (SEQ ID NO: 17) | (SEQ ID NO: 558) |
| 138 | CCND2 | TTTTAGAGTGGAGAAGAG |
| | (SEQ ID NO: 17) | (SEQ ID NO: 559) |
| 139 | CCND2 | GGTAGTTTCGAGGTTTTG |
| | (SEQ ID NO: 17) | (SEQ ID NO: 560) |
| 140 | CCND2 | GGTAGTTTTGAGGTTTTG |
| | (SEQ ID NO: 17) | (SEQ ID NO: 561) |
| 141 | CDH1 | AGGGGGTGCGTGGTTGTA |
| | (SEQ ID NO: 18) | (SEQ ID NO: 562) |
| 142 | CDH1 | AGGGGGTGTGTGGTTGTA |
| | (SEQ ID NO: 18) | (SEQ ID NO: 563) |
| 143 | CDH1 | AGTTTCGACGTTATTGAG |
| | (SEQ ID NO: 18) | (SEQ ID NO: 564) |
| 144 | CDH1 | AGTTTTGATGTTATTGAG |
| | (SEQ ID NO: 18) | (SEQ ID NO: 565) |
| 145 | CDH1 | AGAGGTTGCGGTTTTAAG |
| | (SEQ ID NO: 18) | (SEQ ID NO: 56) |
| 146 | CDH1 | AGAGGTTGTGGTTTTAAG |
| | (SEQ ID NO: 18) | (SEQ ID NO: 567) |
| 147 | CDH1 | AGGGGATTCGGGGTATTT |
| | (SEQ ID NO: 18) | (SEQ ID NO: 568) |
| 148 | CDH1 | AGGGGATTTGGGGTATTT |
| | (SEQ ID NO: 18) | (SEQ ID NO: 569) |
| 149 | CDKN1B | AAGAGAAACGTTGGAATA |
| | (SEQ ID NO: 19) | (SEQ ID NO: 570) |
| 150 | CDKN1B | AAGAGAAATGTTGGAATA |
| | (SEQ ID NO: 19) | (SEQ ID NO: 571) |
| 151 | CDKN1B | TTTGATTTCGAGGGGAGT |
| | (SEQ ID NO: 19) | (SEQ ID NO: 914) |
| 152 | CDKN1B | TTTGATTTTGAGGGGAGT |
| | (SEQ ID NO: 19) | (SEQ ID NO: 915) |
| 153 | CDKN1B | GTATTTGGCGGTTGGATT |
| | (SEQ ID NO: 19) | (SEQ ID NO: 572) |
| 154 | CDKN1B | GTATTTGGTGGTTGGATT |
| | (SEQ ID NO: 19) | (SEQ ID NO: 573) |
| 155 | CDKN1B | TATAATTTCGGGAAAGAA |
| | (SEQ ID NO: 19) | (SEQ ID NO: 574) |
| 156 | CDKN1B | TATAATTTTGGGAAAGAA |
| | (SEQ ID NO: 19) | (SEQ ID NO: 575) |
| 157 | CDKN2a | AGAGTGAACGTATTTAAA |
| | (SEQ ID NO: 20) | (SEQ ID NO: 576) |
| 158 | CDKN2a | AGAGTGAATGTATTTAAA |
| | (SEQ ID NO: 20) | (SEQ ID NO: 577) |
| 159 | CDKN2a | GTTATATTCGTTAAGTGT |
| | (SEQ ID NO: 20) | (SEQ ID NO: 578) |
| 160 | CDKN2a | GTTATATTTGTTAAGTGT |
| | (SEQ ID NO: 20) | (SEQ ID NO: 579) |
| 161 | CDKN2a | TAAGTGTTCGGAGTTAAT |
| | (SEQ ID NO: 20) | (SEQ ID NO: 580) |
| 162 | CDKN2a | TAAGTGTTTGGAGTTAAT |
| | (SEQ ID NO: 20) | (SEQ ID NO: 581) |
| 163 | CDKN2a | GTTAGTATCGGAGGAAGA |
| | (SEQ ID NO: 20) | (SEQ ID NO: 582) |
| 164 | CDKN2a | GTTAGTATTGGAGGAAGA |
| | (SEQ ID NO: 20) | (SEQ ID NO: 583) |
| 165 | CDKN2a | GGAGTTTTCGGTTGATTG |
| | (SEQ ID NO: 20) | (SEQ ID NO: 896) |
| 166 | CDKN2a | GGAGTTTTTGGTTGATTG |
| | (SEQ ID NO: 20) | (SEQ ID NO: 897) |
| 167 | CDKN2a | TTGTTTAACGTATCGAAT |
| | (SEQ ID NO: 20) | (SEQ ID NO: 584) |
| 168 | CDKN2a | TTGTTTAATGTATTGAAT |
| | (SEQ ID NO: 20) | (SEQ ID NO: 585) |
| 169 | CDKN2a | AATAGTTACGGTCGGAGG |
| | (SEQ ID NO: 20) | (SEQ ID NO: 586) |
| 170 | CDKN2a | AATAGTTATGGTTGGAGG |
| | (SEQ ID NO: 20) | (SEQ ID NO: 587) |
| 171 | CDKN2B | ATATTTAGCGAGTAGTGT |
| | (SEQ ID NO: 21) | (SEQ ID NO: 588) |
| 172 | CDKN2B | ATATTTAGTGAGTAGTGT |
| | (SEQ ID NO: 21) | (SEQ ID NO: 589) |
| 173 | CDKN2B | TGGGGAGACGTCGGTTTT |
| | (SEQ ID NO: 21) | (SEQ ID NO: 590) |
| 174 | CDKN2B | TGGGGAGATGTTGGTTTT |
| | (SEQ ID NO: 21) | (SEQ ID NO: 591) |
| 175 | CDKN2B | TTATTGTACGGGGTTTTA |
| | (SEQ ID NO: 21) | (SEQ ID NO: 592) |
| 176 | CDKN2B | TTATTGTATGGGGTTTTA |
| | (SEQ ID NO: 21) | (SEQ ID NO: 593) |
| 177 | CDKN2B | TAGAAGGACGACGGGAGG |
| | (SEQ ID NO: 21) | (SEQ ID NO: 594) |
| 178 | CDKN2B | TAGAAGGATGATGGGAGG |
| | (SEQ ID NO: 21) | (SEQ ID NO: 595) |
| 179 | CDKN2B | AGAGAGTGCGTCGGAGTA |
| | (SEQ ID NO: 21) | (SEQ ID NO: 596) |
| 180 | CDKN2B | AGAGAGTGTGTTGGAGTA |
| | (SEQ ID NO: 21) | (SEQ ID NO: 597) |
| 181 | CD44 | GTGGGGTTCGGAGGTATA |
| | (SEQ ID NO: 22) | (SEQ ID NO: 598) |
| 182 | CD44 | GTGGGGTTTGGAGGTATA |
| | (SEQ ID NO: 22) | (SEQ ID NO: 599) |
| 183 | CD44 | AGGTATTTCGCGATATTT |
| | (SEQ ID NO: 22) | (SEQ ID NO: 600) |
| 184 | CD44 | AGGTATTTTGTGATAGTTT |
| | (SEQ ID NO: 22) | (SEQ ID NO: 601) |
| 185 | CD44 | TTGTTTAGCGGATTTTAG |
| | (SEQ ID NO: 22) | (SEQ ID NO: 602) |
| 186 | CD44 | TTGTTTAGTGGATTTTAG |
| | (SEQ ID NO: 22) | (SEQ ID NO: 603) |
| 187 | CD44 | TGGTGGTACGTAGTTTGG |
| | (SEQ ID NO: 22) | (SEQ ID NO: 604) |
| 188 | CD44 | TGGTGGTATGTAGTTTGG |
| | (SEQ ID NO: 22) | (SEQ ID NO: 605) |
| 189 | CD44 | TGAGTGTTCGTCGTAGTT |
| | (SEQ ID NO: 22) | (SEQ ID NO: 606) |
| 190 | CD44 | TGAGTGTTTGTCGTAGTT |
| | (SEQ ID NO: 22) | (SEQ ID NO: 607) |
| 191 | CSPG2 | AAGATTTTCGGTTAGTTT |
| | (SEQ ID NO: 23) | (SEQ ID NO: 608) |
| 192 | CSPG2 | AAGATTTTTGGTTAGTTT |
| | (SEQ ID NO: 23) | (SEQ ID NO: 609) |
| 193 | CSPG2 | ATGTGATTCGTTTGGGTA |
| | (SEQ ID NO: 23) | (SEQ ID NO: 610) |
| 194 | CSPG2 | ATGTGATTTGTTTGGGTA |
| | (SEQ ID NO: 23) | (SEQ ID NO: 611) |
| 195 | CSPG2 | GGGTAACGTCGAATTTAG |
| | (SEQ ID NO: 23) | (SEQ ID NO: 612) |
| 196 | CSPG2 | GGGTAATGTTGAATTTAG |
| | (SEQ ID NO: 23) | (SEQ ID NO: 613) |
| 197 | CSPG2 | AAAAATTCGCGAGTTTAG |
| | (SEQ ID NO: 23) | (SEQ ID NO: 614) |
| 198 | CSPG2 | AAAAATTTGTGAGTTTAG |
| | (SEQ ID NO: 23) | (SEQ ID NO: 615) |
| 199 | DAPK1 | GTTGGAGTCGAGGTTTGA |
| | (SEQ ID NO: 24) | (SEQ ID NO: 616) |
| 200 | DAPK1 | GTTGGAGTTGAGGTTTGA |
| | (SEQ ID NO: 24) | (SEQ ID NO: 617) |
| 201 | DAPK1 | TTTTTTGTCGGATTGGTG |
| | (SEQ ID NO: 24) | (SEQ ID NO: 618) |
| 202 | DAPK1 | TTTTTTGTTGGATTGGTG |
| | (SEQ ID NO: 24) | (SEQ ID NO: 619) |
| 203 | DAPK1 | GAAGGGAGCGTATTTTAT |
| | (SEQ ID NO: 24) | (SEQ ID NO: 620) |
| 204 | DAPK1 | GAAGGGAGTGTATTTTAT |
| | (SEQ ID NO: 24) | (SEQ ID NO: 621) |
| 205 | DAPK1 | TTGTTTTTCGGAAATTTG |
| | (SEQ ID NO: 24) | (SEQ ID NO: 622) |
| 206 | DAPK1 | TTGTTTTTTGGAAATTTG |
| | (SEQ ID NO: 24) | (SEQ ID NO: 623) |
| 207 | GGT1 | ATAGGTGGCGTTTGGATT |
| | (SEQ ID NO: 25) | (SEQ ID NO: 624) |
| 208 | GGT1 | ATAGGTGGTGTTTGGATT |
| | (SEQ ID NO: 25) | (SEQ ID NO: 625) |
| 209 | GGT1 | GGGTGGTGCGTTGTTGTA |
| | (SEQ ID NO: 25) | (SEQ ID NO: 626) |
| 210 | GGT1 | GGGTGGTGTGTTGTTGTA |
| | (SEQ ID NO: 25) | (SEQ ID NO: 627) |
| 211 | GGT1 | TATATTATCGGTTTTAGG |
| | (SEQ ID NO: 25) | (SEQ ID NO: 628) |
| 212 | GGT1 | TATATTATTGGTTTTAGG |
| | (SEQ ID NO: 25) | (SEQ ID NO: 629) |
| 213 | GGT1 | AGGTTAGACGTTTTGTAT |
| | (SEQ ID NO: 25) | (SEQ ID NO: 630) |
| 214 | GGT1 | AGGTTAGATGTTTTGTAT |
| | (SEQ ID NO: 25) | (SEQ ID NO: 631) |
| 215 | GSTP1 | GGTTTTTTCGGTTAGTTG |
| | (SEQ ID NO: 26) | (SEQ ID NO: 632) |
| 216 | GSTP1 | GGTTTTTTTGGTTAGTTG |
| | (SEQ ID NO: 26) | (SEQ ID NO: 633) |
| 217 | GSTP1 | TTTTAGGGGCGTTTTTTG |
| | (SEQ ID NO: 26) | (SEQ ID NO: 634) |
| 218 | GSTP1 | TTTTAGGGTGTTTTTTTG |
| | (SEQ ID NO: 26) | (SEQ ID NO: 635) |
| 219 | GSTP1 | GTAGTTTTCGTTATTAGT |
| | (SEQ ID NO: 26) | (SEQ ID NO: 636) |
| 220 | GSTP1 | GTAGTTTTTGTTATTAGT |
| | (SEQ ID NO: 26) | (SEQ ID NO: 637) |
| 221 | HIC-1 | ATGATTCGTCGTGGGTTT |
| | (SEQ ID NO: 27) | (SEQ ID NO: 638) |
| 222 | HIC-1 | ATGATTTGTTGTGGGTTT |
| | (SEQ ID NO: 27) | (SEQ ID NO: 639) |
| 223 | HIC-1 | AGGAGATTCGAAAGTTTA |
| | (SEQ ID NO: 27) | (SEQ ID NO: 640) |
| 224 | HIC-1 | AGGAGATTTGAAAGTTTA |
| | (SEQ ID NO: 27) | (SEQ ID NO: 641) |
| 225 | HIC-1 | GGGTTTTACGTGGTTGTT |
| | (SEQ ID NO: 27) | (SEQ ID NO: 642) |
| 226 | HIC-1 | GGGTTTTATGTGGTTGTT |
| | (SEQ ID NO: 27) | (SEQ ID NO: 643) |
| 227 | HIC-1 | TTTTAGAGCGTTAGGGTT |
| | (SEQ ID NO: 27) | (SEQ ID NO: 644) |
| 228 | HIC-1 | TTTTAGAGTGTTAGGGTT |
| | (SEQ ID NO: 27) | (SEQ ID NO: 645) |
| 229 | LAP18 | ATTAAAGGCGATTAAATT |
| | (SEQ ID NO: 28) | (SEQ ID NO: 646) |
| 230 | LAP18 | ATTAAAGGTGATTAAATT |
| | (SEQ ID NO: 28) | (SEQ ID NO: 647) |
| 231 | LAP18 | GGTAAGAACGTATATAGT |
| | (SEQ ID NO: 28) | (SEQ ID NO: 648) |
| 232 | LAP18 | GGTAAGAATGTATATAGT |
| | (SEQ ID NO: 28) | (SEQ ID NO: 649) |
| 233 | LAP18 | AGAAATTACGATGATGTT |
| | (SEQ ID NO: 28) | (SEQ ID NO: 650) |
| 234 | LAP18 | AGAAATTATGATGATGTT |
| | (SEQ ID NO: 28) | (SEQ ID NO: 651) |
| 235 | LAP18 | GTGGGTGGCGTATTAGAA |
| | (SEQ ID NO: 28) | (SEQ ID NO: 652) |
| 236 | LAP18 | GTGGGTGGTGTATTAGAA |
| | (SEQ ID NO: 28) | (SEQ ID NO: 653) |
| 237 | LKB1 | GGGTTAAGCGTCGATTAA |
| | (SEQ ID NO: 29) | (SEQ ID NO: 654) |
| 238 | LKB1 | GGGTTAAGTGTTGATTAA |
| | (SEQ ID NO: 29) | (SEQ ID NO: 655) |
| 239 | LKB1 | TAGAGGGTCGGGGATGGT |
| | (SEQ ID NO: 29) | (SEQ ID NO: 656) |
| 240 | LKB1 | TAGAGGGTTGGGGATGGT |
| | (SEQ ID NO: 29) | (SEQ ID NO: 657) |
| 241 | LKB1 | TTTAGGTTCGTAAGTTTA |
| | (SEQ ID NO: 29) | (SEQ ID NO: 658) |
| 242 | LKB1 | TTTAGGTTTGTAAGTTTA |
| | (SEQ ID NO: 29) | (SEQ ID NO: 659) |
| 243 | LKB1 | AGGGAGGTCGTTGGTATT |
| | (SEQ ID NO: 29) | (SEQ ID NO: 912) |
| 244 | LKB1 | AGGGAGGTTGTTGGTATT |
| | (SEQ ID NO: 29) | (SEQ ID NO: 913) |
| 245 | LKB1 | TTAATGAGCGCGTTGTAT |
| | (SEQ ID NO: 29) | (SEQ ID NO: 660) |
| 246 | LKB1 | TTAATGAGTGCGTTGTAT |
| | (SEQ ID NO: 29) | (SEQ ID NO: 661) |
| 247 | LOC51147 | TTTAGTGACGAGAAGGTT |
| | (SEQ ID NO: 30) | (SEQ ID NO: 662) |
| 248 | LOC51147 | TTTAGTGATGAGAAGGTT |
| | (SEQ ID NO: 30) | (SEQ ID NO: 663) |
| 249 | LOC51147 | TTATGAAGCGGTTTTGTG |
| | (SEQ ID NO: 30) | (SEQ ID NO: 664) |
| 250 | LOC51147 | TTATGAAGTGGTTTTGTG |
| | (SEQ ID NO: 30) | (SEQ ID NO: 665) |
| 251 | LOC51147 | GTAGTAGGATCGAGGTTT |
| | (SEQ ID NO: 30) | (SEQ ID NO: 666) |
| 252 | LOC51147 | GTAGTAGGATTGAGGTTT |
| | (SEQ ID NO: 30) | (SEQ ID NO: 667) |
| 253 | LOC51147 | GTTAGAGACGTGTTTTGA |
| | (SEQ ID NO: 30) | (SEQ ID NO: 668) |
| 254 | LOC51147 | GTTAGAGATGTGTTTTGA |
| | (SEQ ID NO: 30) | (SEQ ID NO: 669) |
| 255 | MGMT | TAAGGATACGAGTTATAT |
| | (SEQ ID NO: 31) | (SEQ ID NO: 670) |
| 256 | MGMT | TAAGGATATGAGTTATAT |
| | (SEQ ID NO: 31) | (SEQ ID NO: 671) |
| 257 | MGMT | TTGGAGAGCGGTTGAGTT |
| | (SEQ ID NO: 31) | (SEQ ID NO: 672) |
| 258 | MGMT | TTGGAGAGTGGTTGAGTT |
| | (SEQ ID NO: 31) | (SEQ ID NO: 673) |
| 259 | MGMT | TAGGTTATCGGTGATTGT |
| | (SEQ ID NO: 31) | (SEQ ID NO: 890) |
| 260 | MGMT | TAGGTTATTGGTGATTGT |
| | (SEQ ID NO: 31) | (SEQ ID NO: 891) |
| 261 | MGMT | AGTAGGATCGGGATTTTT |
| | (SEQ ID NO: 31) | (SEQ ID NO: 674) |
| 262 | MGMT | AGTAGGATTGGGATTTTT |
| | (SEQ ID NO: 31) | (SEQ ID NO: 675) |
| 263 | MLH1 | TTGAGAAGCGTTAAGTAT |
| | (SEQ ID NO: 32) | (SEQ ID NO: 676) |
| 264 | MLH1 | TTGAGAAGTGTTAAGTAT |
| | (SEQ ID NO: 32) | (SEQ ID NO: 677) |
| 265 | MLH1 | TTAGGTAGCGGGTAGTAG |
| | (SEQ ID NO: 32) | (SEQ ID NO: 678) |
| 266 | MLH1 | TTAGGTAGTGGGTAGTAG |
| | (SEQ ID NO: 32) | (SEQ ID NO: 679) |
| 267 | MLH1 | GTAGTAGTCGTTTTAGGG |
| | (SEQ ID NO: 32) | (SEQ ID NO: 680) |
| 268 | MLH1 | GTAGTAGTTGTTTTAGGG |
| | (SEQ ID NO: 32) | (SEQ ID NO: 681) |
| 269 | MLH1 | ATAGTTGTCGTTGAAGGG |
| | (SEQ ID NO: 32) | (SEQ ID NO: 682) |
| 270 | MLH1 | ATAGTTGTTGTTGAAGGG |
| | (SEQ ID NO: 32) | (SEQ ID NO: 683) |
| 271 | MLH1 | GGGTTATTCGGCGGTTGG |
| | (SEQ ID NO: 32) | (SEQ ID NO: 684) |
| 272 | MLH1 | GGGTTATTTGGTGGTTGG |
| | (SEQ ID NO: 32) | (SEQ ID NO: 685) |
| 273 | MNCA9 | TAAAAGGGCGTTTTGTGA |
| | (SEQ ID NO: 33) | (SEQ ID NO: 686) |
| 274 | MNCA9 | TAAAAGGGTGTTTTGTGA |
| | (SEQ ID NO: 33) | (SEQ ID NO: 687) |
| 275 | MNCA9 | TTAATGTACGTATAGTTC |
| | (SEQ ID NO: 33) | (SEQ ID NO: 688) |
| 276 | MNCA9 | TTAATGTATGTATAGTTC |
| | (SEQ ID NO: 33) | (SEQ ID NO: 689) |
| 277 | MNCA9 | GTATATATCGTGTGTTGG |
| | (SEQ ID NO: 33) | (SEQ ID NO: 690) |
| 278 | MNCA9 | GTATATATTGTGTGTTGG |
| | (SEQ ID NO: 33) | (SEQ ID NO: 691) |
| 279 | MNCA9 | TAGTTAGTCGTATGGTTT |
| | (SEQ ID NO: 33) | (SEQ ID NO: 692) |
| 280 | MNCA9 | TAGTTAGTTGTATGGTTT |
| | (SEQ ID NO: 33) | (SEQ ID NO: 693) |
| 281 | MYC | TTAGAGTGTTCGGTTGTT |
| | (SEQ ID NO: 34) | (SEQ ID NO: 694) |
| 282 | MYC | TTAGAGTGTTTGGTTGTT |
| | (SEQ ID NO: 34) | (SEQ ID NO: 695) |
| 283 | MYC | AGGATTTTCGAGTTGTGT |
| | (SEQ ID NO: 34) | (SEQ ID NO: 696) |
| 284 | MYC | AGGATTTTTGAGTTGTGT |
| | (SEQ ID NO: 34) | (SEQ ID NO: 697) |
| 285 | MYC | GAGGGATCGCGTTGAGTA |
| | (SEQ ID NO: 34) | (SEQ ID NO: 900) |
| 286 | MYC | GAGGGATTGTGTTGAGTA |
| | (SEQ ID NO: 34) | (SEQ ID NO: 901) |
| 287 | MYC | AATTTTAGCGAGAGGTAG |
| | (SEQ ID NO: 34) | (SEQ ID NO: 698) |
| 288 | MYC | AATTTTAGTGAGAGGTAG |
| | (SEQ ID NO: 34) | (SEQ ID NO: 699) |
| 289 | MYC | TTGTGGGCGTTTTGGGAA |
| | (SEQ ID NO: 34) | (SEQ ID NO: 700) |
| 290 | MYC | TTGTGGGTGTTTTGGGAA |
| | (SEQ ID NO: 34) | (SEQ ID NO: 701) |
| 291 | N33 | GTGAATCGGATGTTTTGT |
| | (SEQ ID NO: 35) | (SEQ ID NO: 702) |
| 292 | N33 | GTGAATTGGATGTTTTGT |
| | (SEQ ID NO: 35) | (SEQ ID NO: 703) |
| 293 | N33 | GTTTAGTTAGCGGGTTTT |
| | (SEQ ID NO: 35) | (SEQ ID NO: 704) |
| 294 | N33 | GTTTAGTTAGTGGGTTTT |
| | (SEQ ID NO: 35) | (SEQ ID NO: 705) |
| 295 | N33 | GTTTTGTCGCGATGGGGG |
| | (SEQ ID NO: 35) | (SEQ ID NO: 706) |
| 296 | N33 | GTTTTGTTGTGATGGGGG |
| | (SEQ ID NO: 35) | (SEQ ID NO: 707) |
| 297 | N33 | ATTTAGTTCGGGGGAGGA |
| | (SEQ ID NO: 35) | (SEQ ID NO: 708) |
| 298 | N33 | ATTTAGTTTGGGGGAGGA |
| | (SEQ ID NO: 35) | (SEQ ID NO: 709) |
| 299 | PAX6 | TTTTTGGTCGTAGGGTTG |
| | (SEQ ID NO: 36) | (SEQ ID NO: 710) |
| 300 | PAX6 | TTTTTGGTTGTAGGGTTG |
| | (SEQ ID NO: 36) | (SEQ ID NO: 711) |
| 301 | PAX6 | TATTGTTTCGGTTGTTAG |
| | (SEQ ID NO: 36) | (SEQ ID NO: 902) |
| 302 | PAX6 | TATTGTTTTGGTTGTTAG |
| | (SEQ ID NO: 36) | (SEQ ID NO: 903) |
| 303 | PAX6 | TTTAGGTCGCGTAGATTT |
| | (SEQ ID NO: 36) | (SEQ ID NO: 712) |
| 304 | PAX6 | TTTAGGTTGTGTAGATTT |
| | (SEQ ID NO: 36) | (SEQ ID NO: 713) |
| 305 | PAX6 | AGAGTTTAGCGTATTTTT |
| | (SEQ ID NO: 36) | (SEQ ID NO: 714) |
| 306 | PAX6 | AGAGTTTAGTGTATTTTT |
| | (SEQ ID NO: 36) | (SEQ ID NO: 715) |
| 307 | PGR | AAGGAGTCGCGTGTTATT |
| | (SEQ ID NO: 37) | (SEQ ID NO: 716) |
| 308 | PGR | AAGGAGTTGTGTGTTATT |
| | (SEQ ID NO: 37) | (SEQ ID NO: 717) |
| 309 | PGR | TTAAGTGTCGGATTTGTG |
| | (SEQ ID NO: 37) | (SEQ ID NO: 718) |
| 310 | PGR | TTAAGTGTTGGATTTGTG |
| | (SEQ ID NO: 37) | (SEQ ID NO: 719) |
| 311 | PGR | TTAGTTTTCGGATAGAAG |
| | (SEQ ID NO: 37) | (SEQ ID NO: 720) |
| 312 | PGR | TTAGTTTTTGGATAGAAG |
| | (SEQ ID NO: 37) | (SEQ ID NO: 721) |
| 313 | PGR | GGGATAAACGATAGTTAT |
| | (SEQ ID NO: 37) | (SEQ ID NO: 722) |
| 314 | PGR | GGGATAAATGATAGTTAT |
| | (SEQ ID NO: 37) | (SEQ ID NO: 723) |
| 315 | PTEN | GGATTTTGCGTTCGTATT |
| | (SEQ ID NO: 38) | (SEQ ID NO: 724) |
| 316 | PTEN | GGATTTTGTGTTTGTATT |
| | (SEQ ID NO: 38) | (SEQ ID NO: 725) |
| 317 | PTEN | AGAGTTATCGTTTTGTTT |
| | (SEQ ID NO: 38) | (SEQ ID NO: 726) |
| 318 | PTEN | AGAGTTATTGTTTTGTTT |
| | (SEQ ID NO: 38) | (SEQ ID NO: 727) |
| 319 | PTEN | TGATGTGGCGGGATTTTT |
| | (SEQ ID NO: 38) | (SEQ ID NO: 728) |
| 320 | PTEN | TGATGTGGTGGGATTTTT |
| | (SEQ ID NO: 38) | (SEQ ID NO: 729) |
| 321 | PTEN | TTTTTATGCGTTGCGGTA |
| | (SEQ ID NO: 38) | (SEQ ID NO: 730) |
| 322 | PTEN | TTTTTATGTGTTGTGGTA |
| | (SEQ ID NO: 38) | (SEQ ID NO: 731) |
| 323 | RARB | TAGTAGTTCGGGTAGGGT |
| | (SEQ ID NO: 39) | (SEQ ID NO: 906) |
| 324 | RARB | TAGTAGTTTGGGTAGGGT |
| | (SEQ ID NO: 39) | (SEQ ID NO: 907) |
| 325 | RARB | GGGTTTATCGAAAGTTTA |
| | (SEQ ID NO: 39) | (SEQ ID NO: 732) |
| 326 | RARB | GGGTTTATTGAAAGTTTA |
| | (SEQ ID NO: 39) | (SEQ ID NO: 733) |
| 327 | RARB | TTTTTATGCGAGTTGTTT |
| | (SEQ ID NO: 39) | (SEQ ID NO: 734) |
| 328 | RARB | TTTTTATGTGAGTTGTTT |
| | (SEQ ID NO: 39) | (SEQ ID NO: 735) |
| 329 | RARB | TTGGGTATCGTCGGGGTA |
| | (SEQ ID NO: 39) | (SEQ ID NO: 736) |
| 330 | RARB | TTGGGTATTGTTGGGGTA |
| | (SEQ ID NO: 39) | (SEQ ID NO: 737) |
| 331 | SFN | ATAGAGTTCGGTATTGGT |
| | (SEQ ID NO: 40) | (SEQ ID NO: 738) |
| 332 | SFN | ATAGAGTTTGGTATTGGT |
| | (SEQ ID NO: 40) | (SEQ ID NO: 739) |
| 333 | SFN | GAGTAGGTCGAACGTTAT |
| | (SEQ ID NO: 40) | (SEQ ID NO: 884) |
| 334 | SFN | GAGTAGGTTGAATGTTAT |
| | (SEQ ID NO: 40) | (SEQ ID NO: 885) |
| 335 | SFN | AAAAGTAACGAGGAGGGT |
| | (SEQ ID NO: 40) | (SEQ ID NO: 888) |
| 336 | SFN | AAAAGTAATGAGGAGGGT |
| | (SEQ ID NO: 40) | (SEQ ID NO: 889) |
| 337 | SFN | TTTTAGGGCGTGTGCGAT |
| | (SEQ ID NO: 40) | (SEQ ID NO: 740) |
| 338 | SFN | TTTTAGGGTGTGTGTGAT |
| | (SEQ ID NO: 40) | (SEQ ID NO: 741) |
| 339 | S100A2 | TTTAATTGCGGTTGTGTG |
| | (SEQ ID NO: 41) | (SEQ ID NO: 742) |
| 340 | S100A2 | TTTAATTGTGGTTGTGTG |
| | (SEQ ID NO: 41) | (SEQ ID NO: 743) |
| 341 | S100A2 | TATATAGGCGTATGTATG |
| | (SEQ ID NO: 41) | (SEQ ID NO: 744) |
| 342 | S100A2 | TATATAGGTGTATGTATG |
| | (SEQ ID NO: 41) | (SEQ ID NO: 745) |
| 343 | S100A2 | TATGTATACGAGTATTGG |
| | (SEQ ID NO: 41) | (SEQ ID NO: 746) |
| 344 | S100A2 | TATGTATATGAGTATTGG |
| | (SEQ ID NO: 41) | (SEQ ID NO: 747) |
| 345 | S100A2 | AGTTTTAGCGTGTGTTTA |
| | (SEQ ID NO: 41) | (SEQ ID NO: 748) |
| 346 | S100A2 | AGTTTTAGTGTGTGTTTA |
| | (SEQ ID NO: 41) | (SEQ ID NO: 749) |
| 347 | TFF1 | GATAGAGACGTGTATAGT |
| | (SEQ ID NO: 42) | (SEQ ID NO: 750) |
| 348 | TFF1 | GATAGAGATGTGTATAGT |
| | (SEQ ID NO: 42) | (SEQ ID NO: 751) |
| 349 | TFF1 | TGGTTTTTCGTGAAAGAT |
| | (SEQ ID NO: 42) | (SEQ ID NO: 752) |
| 350 | TFF1 | TGGTTTTTTGTGAAAGAT |
| | (SEQ ID NO: 42) | (SEQ ID NO: 753) |
| 351 | TFF1 | TTGGTTTTCGGTATTTTG |
| | (SEQ ID NO: 42) | (SEQ ID NO: 754) |
| 352 | TFF1 | TTGGTTTTTGGTATTTTG |
| | (SEQ ID NO: 42) | (SEQ ID NO: 755) |
| 353 | TGFBR2 | ATTTGGAGCGAGGAATTT |
| | (SEQ ID NO: 43) | (SEQ ID NO: 756) |
| 354 | TGFBR2 | ATTTGGAGTGAGGAATTT |
| | (SEQ ID NO: 43) | (SEQ ID NO: 757) |
| 355 | TGFBR2 | TTGAAAGTCGGTTAAAGT |
| | (SEQ ID NO: 43) | (SEQ ID NO: 758) |
| 356 | TGFBR2 | TTGAAAGTTGGTTAAAGT |
| | (SEQ ID NO: 43) | (SEQ ID NO: 759) |
| 357 | TGFBR2 | AAAGTTTTCGGAGGGGTT |
| | (SEQ ID NO: 43) | (SEQ ID NO: 760) |
| 358 | TGFBR2 | AAAGTTTTTGGAGGGGTT |
| | (SEQ ID NO: 43) | (SEQ ID NO: 761) |
| 359 | TGFBR2 | GGTAGTTACGAGAGAGTT |
| | (SEQ ID NO: 43) | (SEQ ID NO: 762) |
| 360 | TGFBR2 | GGTAGTTATGAGAGAGTT |
| | (SEQ ID NO: 43) | (SEQ ID NO: 763) |
| 361 | TGFBR2 | GTTGGACGTCGAGGAGAG |
| | (SEQ ID NO: 43) | (SEQ ID NO: 764) |
| 362 | TGFBR2 | GTTGGATGTTGAGGAGAG |
| | (SEQ ID NO: 43) | (SEQ ID NO: 765) |
| 363 | TIMP3 | AGGTTTTTCGTTGGAGAA |
| | (SEQ ID NO: 44) | (SEQ ID NO: 766) |
| 364 | TIMP3 | AGGTTTTTTGTTGGAGAA |
| | (SEQ ID NO: 44) | (SEQ ID NO: 767) |
| 365 | TIMP3 | GAAAATATCGGTATTTTG |
| | (SEQ ID NO: 44) | (SEQ ID NO: 768) |
| 366 | TIMP3 | GAAAATATTGGTATTTTG |
| | (SEQ ID NO: 44) | (SEQ ID NO: 769) |
| 367 | TIMP3 | ATGTGGGGCGCGGGGATA |
| | (SEQ ID NO: 44) | (SEQ ID NO: 770) |
| 368 | TIMP3 | ATGTGGGGTGTGGGGATA |
| | (SEQ ID NO: 44) | (SEQ ID NO: 771) |
| 369 | TIMP3 | GGGATAAGCGAATTTTTT |
| | (SEQ ID NO: 44) | (SEQ ID NO: 772) |
| 370 | TIMP3 | GGGATAAGTGAATTTTTT |
| | (SEQ ID NO: 44) | (SEQ ID NO: 773) |
| 371 | VHL | TTTATAAGCGTGATGATT |
| | (SEQ ID NO: 45) | (SEQ ID NO: 774) |
| 372 | VHL | TTTATAAGTGTGATGATT |
| | (SEQ ID NO: 45) | (SEQ ID NO: 775) |
| 373 | VHL | GGTGTTTTCGTGTGAGAT |
| | (SEQ ID NO: 45) | (SEQ ID NO: 916) |
| 374 | VHL | GGTGTTTTTGTGTGAGAT |
| | (SEQ ID NO: 45) | (SEQ ID NO: 917) |
| 375 | VHL | GTATATTGCGCGTTTGAT |
| | (SEQ ID NO: 45) | (SEQ ID NO: 776) |
| 376 | VHL | GTATATTGTGTGTTTGAT |
| | (SEQ ID NO: 45) | (SEQ ID NO: 777) |
| 377 | CDKN1C | ATGAAGAACGGTTAAGGG |
| | (SEQ ID NO: 46) | (SEQ ID NO: 892) |
| 378 | CDKN1C | ATGAAGAATGGTTAAGGG |
| | (SEQ ID NO: 46) | (SEQ ID NO: 893) |
| 379 | CDKN1C | TTAAGTTACGGTTATTAG |
| | (SEQ ID NO: 46) | (SEQ ID NO: 778) |
| 380 | CDKN1C | TTAAGTTATGGTTATTAG |
| | (SEQ ID NO: 46) | (SEQ ID NO: 779) |
| 381 | CDKN1C | TTAGTGTTCGTTTGGAAT |
| | (SEQ ID NO: 46) | (SEQ ID NO: 780) |
| 382 | CDKN1C | TTAGTGTTTGTTTGGAAT |
| | (SEQ ID NO: 46) | (SEQ ID NO: 781) |
| 383 | CAV1 | TTGGTATCGTTGAAGAAT |
| | (SEQ ID NO: 47) | (SEQ ID NO: 782) |
| 384 | CAV1 | TTGGTATTGTTGAAGAAT |
| | (SEQ ID NO: 47) | (SEQ ID NO: 783) |
| 385 | CAV1 | TTTTTGTCGCGGGAATTT |
| | (SEQ ID NO: 47) | (SEQ ID NO: 784) |
| 386 | CAV1 | TTTTTGTTGTGGGAATTT |
| | (SEQ ID NO: 47) | (SEQ ID NO: 785) |
| 387 | CAV1 | TAGATTCGGAGGTAGGTA |
| | (SEQ ID NO: 47) | (SEQ ID NO: 786) |
| 388 | CAV1 | TAGATTTGGAGGTAGGTA |
| | (SEQ ID NO: 47) | (SEQ ID NO: 787) |
| 389 | CAV1 | GAAGTGTTCGTTTTTGTT |
| | (SEQ ID NO: 47) | (SEQ ID NO: 788) |
| 390 | CAV1 | GAAGTGTTTGTTTTTGTT |
| | (SEQ ID NO: 47) | (SEQ ID NO: 789) |
| 391 | CDH13 | TTGTTTAGCGTGATTTGT |
| | (SEQ ID NO: 48) | (SEQ ID NO: 790) |
| 392 | CDH13 | TTGTTTAGTGTGATTTGT |
| | (SEQ ID NO: 48) | (SEQ ID NO: 791) |
| 393 | CDH13 | ATGTAAAACGAGGGAGCG |
| | (SEQ ID NO: 48) | (SEQ ID NO: 792) |
| 394 | CDH13 | ATGTAAAATGAGGGAGTG |
| | (SEQ ID NO: 48) | (SEQ ID NO: 887) |
| 395 | CDH13 | AAGGAATTCGTTTTGTAA |
| | (SEQ ID NO: 48) | (SEQ ID NO: 792) |
| 396 | CDH13 | AAGGAATTTGTTTTGTAA |
| | (SEQ ID NO: 48) | (SEQ ID NO: 793) |
| 397 | CDH13 | AATGTTTTCGTGATGTTG |
| | (SEQ ID NO: 48) | (SEQ ID NO: 794) |
| 398 | CDH13 | AATGTTTTTGTGATGTTG |
| | (SEQ ID NO: 48) | (SEQ ID NO: 795) |
| 399 | NDRG1 | GAGTAGGACGGTGTTAAG |
| | (SEQ ID NO: 49) | (SEQ ID NO: 796) |
| 400 | NDRG1 | GAGTAGGATGGTGTTAAG |
| | (SEQ ID NO: 49) | (SEQ ID NO: 797) |
| 401 | NDRG1 | AAATTTAACGTTGGGTAG |
| | (SEQ ID NO: 49) | (SEQ ID NO: 498) |
| 402 | NDRG1 | AAATTTAATGTTGGGTAG |
| | (SEQ ID NO: 49) | (SEQ ID NO: 799) |
| 403 | NDRG1 | GATAATGACGGTGTTAGT |
| | (SEQ ID NO: 49) | (SEQ ID NO: 800) |
| 404 | NDRG1 | GATAATGATGGTGTTAGT |
| | (SEQ ID NO: 49) | (SEQ ID NO: 801) |
| 405 | NDRG1 | TGGTTGTACGTTAGGAGT |
| | (SEQ ID NO: 49) | (SEQ ID NO: 802) |
| 406 | NDRG1 | TGGTTGTATGTTAGGAGT |
| | (SEQ ID NO: 49) | (SEQ ID NO: 803) |
| 407 | NDRG1 | GTTTTTATCGGGTTTACG |
| | (SEQ ID NO: 49) | (SEQ ID NO: 804) |
| 408 | PTGS2 | GTTTTTATTGGGTTATG |
| | (SEQ ID NO: 50) | (SEQ ID NO: 805) |
| 409 | PTGS2 | AGTTATTTCGTTATATGG |
| | (SEQ ID NO: 50) | (SEQ ID NO: 806) |
| 410 | PTGS2 | AGTTATTTTGTTATATGG |
| | (SEQ ID NO: 50) | (SEQ ID NO: 807) |
| 411 | PTGS2 | TTGGTTTTCGGAAGCGTT |
| | (SEQ ID NO: 50) | (SEQ ID NO: 910) |
| 412 | PTGS2 | TTGGTTTTTGGAAGTGTT |
| | (SEQ ID NO: 50) | (SEQ ID NO: 911) |
| 413 | PTGS2 | AAAGATTGCGAAGAAGAA |
| | (SEQ ID NO: 50) | (SEQ ID NO: 808) |
| 414 | PTGS2 | AAAGATTGTGAAGAAGAA |
| | (SEQ ID NO: 50) | (SEQ ID NO: 809) |
| 415 | PTGS2 | ATATTTGGCGGAAATTTG |
| | (SEQ ID NO: 50) | (SEQ ID NO: 810) |
| 416 | PTGS2 | ATATTTGGTGGAAATTTG |
| | (SEQ ID NO: 50) | (SEQ ID NO: 811) |
| 417 | THBS1 | TTATAAAACGGGTTTAGT |
| | (SEQ ID NO: 51) | (SEQ ID NO: 812) |
| 418 | THBS1 | TTATAAAATGGGTTTAGT |
| | (SEQ ID NO: 51) | (SEQ ID NO: 813) |
| 419 | THBS1 | AGGTATTTCGGGAGATTA |
| | (SEQ ID NO: 51) | (SEQ ID NO: 814) |
| 420 | THBS1 | AGGTATTTTGGGAGATTA |
| | (SEQ ID NO: 51) | (SEQ ID NO: 815) |
| 421 | THBS1 | GATTAGTTCGTTCGAAAG |
| | (SEQ ID NO: 51) | (SEQ ID NO: 816) |
| 422 | THBS1 | GATTAGTTTGTTTGAAAG |
| | (SEQ ID NO: 51) | (SEQ ID NO: 817) |
| 423 | THBS1 | AGTTTTTGCGTTATTTCG |
| | (SEQ ID NO: 51) | (SEQ ID NO: 818) |
| 424 | THBS1 | AGTTTTTGTGTTATTTTG |
| | (SEQ ID NO: 51) | (SEQ ID NO: 819) |
| 425 | TMEFF2 | GATGTTTTCGGTAATTTA |
| | (SEQ ID NO: 52) | (SEQ ID NO: 820) |
| 426 | TMEFF2 | GATGTTTTTGGTAATTTA |
| | (SEQ ID NO: 52) | (SEQ ID NO: 821) |
| 427 | TMEFF2 | ATAGGTTACGGGTTGGAG |
| | (SEQ ID NO: 52) | (SEQ ID NO: 822) |
| 428 | TMEFF2 | ATAGGTTATGGGTTGGAG |
| | (SEQ ID NO: 52) | (SEQ ID NO: 823) |
| 429 | TMEFF2 | TAAATTTGCGAACGTTTG |
| | (SEQ ID NO: 52) | (SEQ ID NO: 824) |
| 430 | TMEFF2 | TAAATTTGTGAATGTTTG |
| | (SEQ ID NO: 52) | (SEQ ID NO: 825) |
| 431 | TMEFF2 | TGAGGTTTCGTTTTAAGA |
| | (SEQ ID NO: 52) | (SEQ ID NO: 826) |
| 432 | TMEFF2 | TGAGGTTTTGTTTTAAGA |
| | (SEQ ID NO: 52) | (SEQ ID NO: 827) |
| 433 | PLAU | TTGGTTTGCGGTTATTA |
| | (SEQ ID NO: 53) | (SEQ ID NO: 828) |
| 434 | PLAU | TTGGTTTGTGGTTATTTA |
| | (SEQ ID NO: 53) | (SEQ ID NO: 829) |
| 435 | PLAU | GTTATTTACGTGTGTGGA |
| | (SEQ ID NO: 53) | (SEQ ID NO: 830) |
| 436 | PLAU | GTTATTTATGTGTGTGGA |
| | (SEQ ID NO: 53) | (SEQ ID NO: 831) |
| 437 | PLAU | TGTTTATGCGTTTATGGT |
| | (SEQ ID NO: 53) | (SEQ ID NO: 832) |
| 438 | PLAU | TGTTTATGTGTTTATGGT |
| | (SEQ ID NO: 53) | (SEQ ID NO: 833) |
| 439 | PLAU | GGATAAGTCGTGTTTTGA |
| | (SEQ ID NO: 53) | (SEQ ID NO: 834) |
| 440 | PLAU | GGATAAGTTGTGTTTTGA |
| | (SEQ ID NO: 53) | (SEQ ID NO: 835) |
| 441 | TMEFF2 | GTGAAGTTCGTTGTTTTT |
| | (SEQ ID NO: 52) | (SEQ ID NO: 908) |
| 442 | TMEFF2 | GTGAAGTTTGTTGTTTTT |
| | (SEQ ID NO: 52) | (SEQ ID NO: 909) |
| 443 | TMEFF2 | TTGTTAAACGTTTATCGG |
| | (SEQ ID NO: 52) | (SEQ ID NO: 836) |
| 444 | TMEFF2 | TTGTTAAATGTTTATTGG |
| | (SEQ ID NO: 52) | (SEQ ID NO: 837) |
| 445 | TMEFF2 | GAAGAATACGCGTATTTA |
| | (SEQ ID NO: 52) | (SEQ ID NO: 838) |
| 446 | TMEFF2 | GAAGAATATGTGTATTTA |
| | (SEQ ID NO: 52) | (SEQ ID NO: 839) |
| 447 | DNMT1 | TAGTAAATCGTGGAGTTT |
| | (SEQ ID NO: 54) | (SEQ ID NO: 840) |
| 448 | DNMT1 | TAGTAAATTGTGGAGTTT |
| | (SEQ ID NO: 54) | (SEQ ID NO: 841) |
| 449 | DNMT1 | AGTGGGTTCGTTTAAGTT |
| | (SEQ ID NO: 54) | (SEQ ID NO: 842) |
| 450 | DNMT1 | AGTGGGTTTGTTTAAGTT |
| | (SEQ ID NO: 54) | (SEQ ID NO: 843) |
| 451 | DNMT1 | TTTTTACGCGGAGTAGTG |
| | (SEQ ID NO: 54) | (SEQ ID NO: 844) |
| 452 | DNMT1 | TTTTTACGTGGAGTAGTG |
| | (SEQ ID NO: 54) | (SEQ ID NO: 845) |
| 453 | DNMT1 | GAGAGAGGCGATATTTTG |
| | (SEQ ID NO: 54) | (SEQ ID NO: 846) |
| 454 | DNMT1 | GAGAGAGGTGATATTTTG |
| | (SEQ ID NO: 54) | (SEQ ID NO: 847) |
| 455 | ESR1 | AGATATATCGGAGTTTGG |
| | (SEQ ID NO: 55) | (SEQ ID NO: 848) |
| 456 | ESR1 | AGATATTGGAGTTTGG |
| | (SEQ ID NO: 55) | (SEQ ID NO: 849) |
| 457 | ESR1 | GTTTGGTACGGGGTATAT |
| | (SEQ ID NO: 55) | (SEQ ID NO: 850) |
| 458 | ESR1 | GTTTGGTATGGGGTATAT |
| | (SEQ ID NO: 55) | (SEQ ID NO: 851) |
| 459 | ESR1 | TTAGTAGCGACGATAAGT |
| | (SEQ ID NO: 55) | (SEQ ID NO: 852) |
| 460 | ESR1 | TTAGTAGTGATGATAAGT |
| | (SEQ ID NO: 55) | (SEQ ID NO: 853) |
| 461 | ESR1 | TATGAGTTCGGGAGATTA |
| | (SEQ ID NO: 55) | (SEQ ID NO: 854) |
| 462 | ESR1 | TATGAGTTTGGGAGATTA |
| | (SEQ ID NO: 55) | (SEQ ID NO: 855) |
| 463 | ESR1 | TGGAGGTTCGGGAGTTTA |
| | (SEQ ID NO: 55) | (SEQ ID NO: 856) |
| 464 | ESR1 | TGGAGGTTTGGGAGTTTA |
| | (SEQ ID NO: 55) | (SEQ ID NO: 857) |
| 465 | APAF1 | TTTGGTATCGTTTAGAGT |
| | (SEQ ID NO: 56) | (SEQ ID NO: 858) |
| 466 | APAF1 | TTTGGTATTGTTTAGAGT |
| | (SEQ ID NO: 56) | (SEQ ID NO: 859) |
| 467 | APAF1 | GTATGAGTCGTGGTAGGA |
| | (SEQ ID NO: 56) | (SEQ ID NO: 860) |
| 468 | APAF1 | GTATGAGTTGTGGTAGGA |
| | (SEQ ID NO: 56) | (SEQ ID NO: 861) |
| 469 | APAF1 | GTGGATTCGGCGGGATTT |
| | (SEQ ID NO: 56) | (SEQ ID NO: 862) |
| 470 | APAF1 | GTGGATTTGGTGGGATTT |
| | (SEQ ID NO: 56) | (SEQ ID NO: 863) |
| 471 | APAF1 | TTTAGAGGCGGAGAAGAA |
| | (SEQ ID NO: 56) | (SEQ ID NO: 864) |
| 472 | APAF1 | TTTAGAGGTGGAGAAGAA |
| | (SEQ ID NO: 56) | (SEQ ID NO: 865) |
| 473 | APAF1 | GAAGAGGTAGCGAGTGGA |
| | (SEQ ID NO: 56) | (SEQ ID NO: 866) |
| 474 | APAF1 | GAAGAGGTAGTGAGTGGA |
| | (SEQ ID NO: 56) | (SEQ ID NO: 867) |
| 475 | HOXA5 | AGTTAGTCGGGTTTTAAG |
| | (SEQ ID NO: 57) | (SEQ ID NO: 868) |
| 476 | HOXA5 | AGTTAGTTGGGTTTTAAG |
| | (SEQ ID NO: 57) | (SEQ ID NO: 869) |
| 477 | HOXA5 | TTATAGGGTTCGGTTTTT |
| | (SEQ ID NO: 57) | (SEQ ID NO: 870) |
| 478 | HOXA5 | TTATAGGGTTTGGTTTTT |
| | (SEQ ID NO: 57) | (SEQ ID NO: 871) |
| 479 | HOXA5 | TTTTAAGGCGAGGTTAAA |
| | (SEQ ID NO: 57) | (SEQ ID NO: 872) |
| 480 | HOXA5 | TTTTAAGGTGAGGTTAAA |
| | (SEQ ID NO: 57) | (SEQ ID NO: 873) |
| 481 | HOXA5 | ATGATAGGCGTTTATTAA |
| | (SEQ ID NO: 57) | (SEQ ID NO: 874) |
| 482 | HOXA5 | ATGATAGGTGTTTATTAA |
| | (SEQ ID NO: 57) | (SEQ ID NO: 875) |
| 483 | RASSF1 | GTAGTTTTCGAGAATGTT |
| | (SEQ ID NO: 58) | (SEQ ID NO: 876) |
| 484 | RASSF1 | GTAGTTTTTGAGAATGTT |
| | (SEQ ID NO: 58) | (SEQ ID NO: 877) |
| 485 | RASSF1 | GGAAATCGGTAATTAGAA |
| | (SEQ ID NO: 58) | (SEQ ID NO: 878) |
| 486 | RASSF1 | GGAAATTGGTAATTAGAA |
| | (SEQ ID NO: 58) | (SEQ ID NO: 879) |
| 487 | RASSF1 | TTTGTGTCGTCGGGAAAT |
| | (SEQ ID NO: 58) | (SEQ ID NO: 880) |
| 488 | RASSF1 | TTTGTGTTGTTGGGAAAT |
| | (SEQ ID NO: 58) | (SEQ ID NO: 881) |
| 489 | RASSF1 | TAGTTTTCGCGTAGAATT |
| | (SEQ ID NO: 58) | (SEQ ID NO: 882) |
| 490 | RASSF1 | TAGTTTTTGTGTAGAATT |
| | (SEQ ID NO: 58) | (SEQ ID NO: 883) |

TABLE 3

Oligonucleotides used in differentiation between
adenocarcinoma and adjacent lung tissue.

| | | |
|---|---|---|
| No: | Gene | Oligo: |

| | | |
|---|---|---|
| 232:1184A | SFN | GAGTAGGTCGACGTTAT |
| | (SEQ ID NO: 40) | (SEQ ID NO: 884) |
| 232:1184B | SFN | GAGTAGGTTGAATGTTAT |
| | (SEQ ID NO: 40) | (SEQ ID NO: 885) |
| 383:1452A | CDH13 | ATGTAAAACGAGGGAGCG |
| | (SEQ ID NO: 48) | (SEQ ID NO: 886) |
| 383:1452B | CDH13 | ATGTAAAATGAGGGAGTG |
| | (SEQ ID N0: 48) | (SEQ ID NO: 887) |
| 232:1346A | SFN | AAAAGTAACGAGGAGGGT |
| | (SEQ ID NO: 40) | (SEQ ID NO: 888) |
| 232:1346B | SFN | AAAAGTAATGAGGAGGGT |
| | (SEQ ID NO: 40) | (SEQ ID NO: 889) |
| 153:374A | MGMT | TAGGTTATCGGTGATTGT |
| | (SEQ ID NO: 31) | (SEQ ID NO: 890) |
| 153:374B | MGMT | TAGGTTATTGGTGATTGT |
| | (SEQ ID NO: 31) | (SEQ ID NO: 891) |
| 350:697A | CDKN1C | ATGAAGAACGGTTAAGGG |
| | (SEQ ID NO: 46) | (SEQ ID NO: 892) |
| 350:697B | CDKN1C | ATGAAGAATGGTTAAGGG |
| | (SEQ ID NO: 46) | SE ID NO: 893) |

TABLE 4

Oligonucleotides used in differentiation between
sauamous cell carcinoma and lung tissue.

| | | |
|---|---|---|
| No: | Gene | Oligo: |

| | | |
|---|---|---|
| 401:40A | HLA-F | TATTTGGGCGGGTGAGTGT |
| | (SEQ ID NO: 10) | (SEQ ID NO: 894) |
| 401:40B | HLA-F | TATTTGGGTGGGTGAGTG |
| | (SEQ ID NO: 10) | (SEQ ID NO: 895) |
| 2035:2074A | CDKN2a | GGAGTTTTCGGTTGATTG |
| | (SEQ ID NO: 20) | (SEQ ID NO: 896) |
| 2035:2074B | CDKN2a | GGAGTTTTTGGTTGATTG |
| | (SEQ ID NO: 20) | (SEQ ID NO: 897) |
| 130:165A | GPIb beta | TTTGAGAGCGGGTGGGAG |
| | (SEQ ID NO: 7) | (SEQ ID NO: 898) |
| 130:165B | GPBb beta | TTTGAGAGTGGGTGGGAG |
| | (SEQ ID NO: 7) | (SEQ ID NO: 899) |
| 2172:1805A | MYC | GAGGGATCGCGTTGAGTA |
| | (SEQ ID NO: 34) | (SEQ ID NO: 900) |
| 2172:1805B | MYC | GAGGGATTGTGTTGAGTA |
| | (SEQ ID NO: 34) | (SEQ ID NO: 901) |
| 2191:310A | PAX6 | TATTGTTTCGGTTGTTAG |
| | (SEQ ID NO: 36) | (SEQ ID NO: 902) |
| 2191:310B | PAX6 | TATTGTTTTGGTTGGTTAG |
| | (SEQ ID NO: 36) | (SEQ ID NO: 903) |
| 130:175A | GPIb beta | GTGGGAGCGGAAGTTTGA |
| | (SEQ ID NO: 7) | (SEQ ID NO: 904) |
| 130:175B | GPIb beta | GTGGGAGTGGAAGTTTGA |
| | (SEQ ID NO: 7) | (SEQ ID NO: 905) |
| 2212:1793A | RARB | TAGTAGTTCGGGTAGGGT |
| | (SEQ ID NO: 39) | (SEQ ID NO: 906) |
| 2212:1793B | RARB | TAGTAGTTTGGGTAGGGT |
| | (SEQ ID NO: 39) | (SEQ ID NO: 907) |
| 2135:868A | LKB1 | AGGGAGGTCGTTGGTATT |
| | (SEQ ID NO: 29) | (SEQ ID NO: 912) |
| 2135:868B | LKB1 | AGGGAGGTTGTTGGTATT |
| | (SEQ ID NO: 29) | (SEQ ID NO: 913) |
| 2034:430A | CDKN1B | TTTGATTTCGAGGGGAGT |
| | (SEQ ID NO: 19) | (SEQ ID NO: 914) |
| 2034:430B | CDKN1B | TTTGATTTTGAGGGGAGT |
| | (SEQ ID NO: 19) | (SEQ ID NO: 915) |
| 2153:374A188 | MGMT | TAGGTTATCGGTGATTGT |
| | | (SEQ ID NO: 890) |
| 2153:374B188 | MGMT | TAGGTTATTGGTGATTGT |
| | | (SEQ ID NO: 891) |

TABLE 5

Oligonucleotides used in differentiation between
adenocarcinoma and squamous cell carcinoma.

| | | |
|---|---|---|
| No: | Gene | Oligo: |

| | | |
|---|---|---|
| 2338:1413A | VHL | GGTGTTTTCGTGTGAGAT |
| | (SEQ ID NO: 45) | (SEQ ID NO: 916) |
| 2338:1413B | VHL | GGTGTTTTTGTGTGAGAT |
| | (SEQ ID NO: 45) | (SEQ ID NO: 917) |
| 2035:2074A | CDKN2a | GGAGTTTTTCGGTTGATTG |
| | (SEQ ID NO: 20) | (SEQ ID NO: 896) |
| 2035:2074B | CDKN2a | GGAGTTTTTGGTTGATTG |
| | (SEQ ID NO: 20) | (SEQ ID NO: 897) |

1. A method for detecting and differentiating between lung cell proliferative disorders associated with at least one gene and/or their regulatory regions from the group comprising MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1 in a subject, said method comprising contacting a target nucleic acid in a biological sample obtained from said subject with at least one reagent or a series of reagents, wherein said reagent or series of reagents, distinguishes between methylated and non methylated CpG dinucleotides within the target nucleic acid.

2. A method according to claim 1 wherein, said method differentiates between at least two members of the following group of medical conditions: adenocarcinoma, squamous cell carcinoma and lung tissue.

3. A method according to claim 1 wherein, said method differentiates between adenocarcinoma and lung tissue.

4. A method according to claim 1 wherein, said method differentiates between squamous cell carcinoma and lung tissue.

5. Use of methods according to claim 1 wherein, said methods are used to differentiate between adenocarcinoma and squamous cell carcinoma.

6. A method according to any one of claims 1 to 5 comprising the following steps:
obtaining a biological sample containing genomic DNA
extracting the genomic DNA
converting cytosine bases in the genomic DNA sample which are unmethylated at the 5-position, by treatment, to uracil or another base which is dissimilar to cytosine in terms of base pairing behaviour;
fragments of the pretreated genomic DNA are amplified, and
identification of the methylation status of one or more cytosine positions.

7. The method according to claim 6, characterised in that the reagent is a solution of bisulfite, hydrogen sulfite or disulfite.

8. The method as recited in claims 6 and 7, characterised in that the amplification is carried out by means of the polymerase chain reaction (PCR).

9. The method as recited in one of the claims 6 through 8, characterised in that the amplification is carried out by means of a heat-resistant DNA polymerase.

10. The method as recited in one of the claims 6 through 9, characterised in that more than ten different fragments having a length of 100-2000 base pairs are amplified.

11. The method as recited in one of claims 6 through 10, wherein the amplification step is carried out using a set of primer oligonucleotides comprising SEQ ID NO: 308 to SEQ ID NO: 427.

12. The method as recited in one of the claims 6 through 11, characterised in that the amplification of several DNA segments is carried out in one reaction vessel.

13. The method as recited in one of claims 6 through 12, characterised in that the amplification step preferentially amplifies DNA which is of particular interest in healthy and/or diseased lung tissues, based on the specific genomic methylation status of lung tissue, as opposed to background DNA.

14. The method according to one of claims 6 through 13, characterised in that the methylation status within at least one gene and/or their regulatory regions from the group comprising MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1 is detected by hybridisation of each amplificate to an oligonucleotide or peptide nucleic acid (PNA)-oligomer.

15. A method according to claim 14, characterised in that the oligonucleotide or peptide nucleic acid (PNA)-oligomer is taken from the group comprising SEQ ID NO: 428 to SEQ ID NO: 917.

16. The method according to claims 6 through 15, characterised in that the amplificates are labelled.

17. The method as recited in claim 16, characterised in that the labels of the amplificates are fluorescence labels.

18. The method as recited in claim 16, characterised in that the labels of the amplificates are radionuclides.

19. The method as recited in claims 16, characterised in that the labels of the amplificates are detachable molecule fragments having a typical mass which are detected in a mass spectrometer.

20. The method as recited in one of the claims 6 through 19, characterised in that the amplificates or fragments of the amplificates are detected in the mass spectrometer.

21. The method as recited in one of the claims 19 and 20, characterised in that the produced fragments have a single positive or negative net charge.

22. The method as recited in one of the claims 19 through 21, characterised in that detection is carried out and visualised by means of matrix assisted laser desorption/ionization mass spectrometry (MALDI) or using electron spray mass spectrometry (ESI).

23. A method according to claims 1 through 5, comprising the following steps:
a) obtaining a biological sample containing genomic DNA
b) extracting the genomic DNA
c) digesting the genomic DNA comprising at least one or more CpGs of the genes MDR1, APOC2, CACNA1G, EGR4, AR, RB1, GP1b beta, MYOD1, WT1, HLA-F, ELK1, APC, ARHI, BCL2, BRCA1, CALCA, CCND2, CDH1, CDKN1B, CDKN2a, CDKN2B, CD44, CSPG2, DAPK1, GGT1, GSTP1, HIC-1, LAP18, LKB1, LOC51147, MGMT, MLH1, MNCA9, MYC, N33, PAX6, PGR, PTEN, RARB, SFN, S100A2, TFF1, TGFBR2, TIMP3, VHL, CDKN1C, CAV1, CDH13, NDRG1, PTGS2, THBS1, TMEFF2, PLAU, DNMT1, ESR1, APAF1, HOXA5 and RASSF1 with one or more methylation sensitive restriction enzymes, and
d) detection of the DNA fragments generated in the digest of step c).

24. A method according to claim 23, wherein the DNA digest is amplified prior to Step d).

25. The method as recited in claim 24, characterised in that the amplification is carried out by means of the polymerase chain reaction (PCR).

26. The method as recited in one of the claims 24 and/or 25, characterised in that the amplification of more than one DNA fragments is carried out in one reaction vessel.

27. The method as recited in one of the claims 24 through 26 characterised in that the polymerase is a heat-resistant DNA polymerase.

28. An isolated nucleic acid of a pretreated genomic DNA according to one of the sequences taken from the group comprising SEQ ID NO: 76 to SEQ ID NO: 307 and sequences complementary thereto.

29. An oligomer, in particular an oligonucleotide or peptide nucleic acid (PNA)-oligomer, said oligomer comprising at least one base sequence of at least 10 nucleotides which hybridises to or is identical to a pretreated genomic DNA according to one of the SEQ ID NO: 76 to SEQ ID NO: 307 according to claim 28.

30. The oligonucleotide as recited in claim 29; wherein the base sequence includes at least one CpG or TpG dinucleotide sequence.

31. The oligonucleotide as recited in claim 30; characterized in that the cytosine of the at least one CpG or TpG dinucleotide is/are located approximately in the middle third of the oligomer.

32. An oligomer, in particular an oligonucleotide or peptide nucleic acid (PNA)-oligomer, according to one of the sequences taken from the group comprising SEQ ID NO: 428 to SEQ ID NO: 917.

33. A set of oligonucleotides, comprising at least two oligonucleotides according to any of claims 29 to 32.

34. A set of oligonucleotides, comprising at least two oligonucleotides according to SEQ ID NO: 884 to 893.

35. One or more isolated nucleic acid(s) taken from the group. according to SEQ ID NO: 59 to 63.

36. A set of oligonucleotides, comprising at least two oligonucleotides according to SEQ ID NO: 894 to 907, 912 to 915, and 890 and 891.

37. One or more isolated nucleic acid(s) taken from the group according to SEQ ID NO: 62, 64 to 70, 73, and 74.

38. A set of oligonucleotides, comprising at least two oligonucleotides according to SEQ ID NO: 896, 897, 916, and 917.

39. One or more isolated nucleic acid(s) taken from the group according to SEQ ID NO: 65 and 75.

40. A set of oligomers, peptide nucleic acid (PNA)-oligomers and/or isolated nucleic acids as recited in claims 33 through 39, comprising oligomers for detecting the methylation state of all CpG dinucleotides within one or more of the sequences according to SEQ ID NO: 1 to SEQ ID NO: 58 and sequences complementary thereto.

41. Use of a set of oligomers or peptide nucleic acid (PNA)-oligomers according to any of claims 29 through 34, 36, and 38 as probes for determining the cytosine methylation state and/or single nucleotide polymorphisms (SNPs) of sequences according to 1 to SEQ ID NO: 58 and sequences complementary thereto.

42. Use of a set of oligonucleotides according to claim 34 or nucleic acid(s) according to claim 35 for the differentiation between adenocarcinoma and lung tissue.

43. Use of a set of oligonucleotides according to claim 36 or nucleic acid(s) according to claim 37 for the differentiation between squamous cell carcinoma and lung tissue.

44. Use of a set of oligonucleotides according to claim 38 or nucleic acid(s) according to claim 39 for the differentiation between adenocarcinoma and squamous cell carcinoma.

45. A set of at least two oligonucleotides or peptide nucleic acid (PNA)-oligomers as recited claim 29, as primer oligonucleotides for the amplification of DNA sequences of one of SEQ ID NO: 76 to SEQ ID NO: 307 according to claim 28 and/or sequences complementary thereto and segments thereof.

46. Use of a pretreated genomic DNA according to claim 28 for the determination of the methylation status of a corresponding genomic DNA and/or detection of single nucleotide polymorphisms (SNP).

47. A set of oligonucleotides or peptide nucleic acid (PNA)-oligomers as recited in claims 33, 34, 36, or 38 characterised in that at least one oligonucleotide is bound to a solid phase.

48. A set of oligonucleotides or peptide nucleic acid (PNA)-oligomers as recited in claims 33, 34, 36 or 38 , characterised in that all members of the set are bound to a solid phase.

49. A method for manufacturing an arrangement of different oligomers or peptide nucleic acid (PNA)-oligomers (array) for analysing diseases associated with the corresponding genomic methylation status of the CpG dinucleotides within one of the SEQ ID NO: 1 to SEQ ID NO: 58 and sequences complementary thereto , wherein at least one oligomer according to any of the claims 33, 34, 36 or 38 is coupled to a solid phase.

50. An arrangement of different oligomers or peptide nucleic acid (PNA)-oligomers (array) obtainable according to claims 47 and 48.

51. An array of different oligonucleotide- and/or PNA-oligomer sequences as recited in claim 50, characterised in that these are arranged on a plane solid phase in the form of a rectangular or hexagonal lattice.

52. A nucleic acid or peptide nucleic acid array for the analysis of lung cell proliferative disorders associated with the methylation state of genes comprising at least one nucleic acid according to one of the preceding claims.

53. The array as recited in and of the claims 50 through 62, characterised in that the solid phase surface is composed of silicon, glass, polystyrene, aluminium, steel, iron, copper, nickel, silver, or gold.

54. A kit comprising a bisulfite (=disulfite, hydrogen sulfite) reagent as well as oligonucleotides and/or PNA-oligomers according to one of the claims 29 through 39.

55. The use of oligonucleotides or peptide nucleic acid (PNA)-oligomers according to SEQ ID NO: 76 to SEQ ID NO: 917 for the detection of a predisposition to differentiation between subclasses, diagnosis, prognosis, treatment and/or monitoring of lung cell proliferative disorders.

56. A DNA sequence according to one of the sequences taken from the group comprising SEQ ID NO: 76 to SEQ ID NO: 307 and sequences complementary thereto for use in the analysis of cytosine methylation within said nucleic acid for the detection of a predisposition to, differentiation between subclasses, diagnosis, prognosis, treatment and/or monitoring of lung cell proliferative disorders.
